(19) United States
(12) Patent Application Publication
(10) Pub. No.: US 2010/0332445 A1
(43) Pub. Date: Dec. 30, 2010

(54) ANALYTE TESTING METHOD AND SYSTEM

(75) Inventors: Pinaki RAY, Fremont, CA (US); Greg Matian, Foster City, CA (US); Aparna Srinivasan, San Jose, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: LifeScan, Inc., Milpitas, CA (US)

(21) Appl. No.: 12/826,659

(22) Filed: Jun. 29, 2010

Related U.S. Application Data
(60) Provisional application No. 61/221,742, filed on Jun. 30, 2009.
(60) Provisional application No. 61/297,553, filed on Jan. 22, 2010.

(51) Int. Cl. G06N 5/02; A61B 5/15; G06F 7/08

(52) U.S. Cl. 706/54; 600/365; 708/202

(57) ABSTRACT

A system and method of detecting a flagged glucose concentration pattern with the use of medians having a common type of flag collected over discrete time periods so that whenever significant differences between the medians arise, the user or a caretaker of a diabetic user is notified.

This application claims the benefits of priority under 35 USC§119 and/or §120 from prior filed U.S. Provisional Application Ser. Nos. 61/221,742 filed on Jun. 30, 2009, and 61/297,553 filed on Jan. 22, 2010, which applications are incorporated by reference in their entirety into this application.

### BACKGROUND

Glucose monitoring is a fact of everyday life for diabetic individuals. The accuracy of such monitoring can significantly affect the health and ultimately the quality of life of the person with diabetes. Generally, a diabetic patient measures blood glucose levels several times a day to monitor and control blood sugar levels. Failure to test blood glucose levels accurately and on a regular basis can result in serious diabetes-related complications, including cardiovascular disease, kidney disease, nerve damage and blindness. There are a number of electronic devices currently available which enable an individual to test the glucose level in a small sample of blood. One such glucose meter is the OneTouch® Profile™ glucose meter, a product which is manufactured by LifeScan.

In addition to glucose monitoring, diabetic individuals often have to maintain tight control over their lifestyle, so that they are not adversely affected by, for example, irregular food consumption or exercise. In addition, a physician dealing with a particular diabetic individual may require detailed information on the lifestyle of the individual to provide effective treatment or modification of treatment for controlling diabetes. Currently, one of the ways of monitoring the lifestyle of an individual with diabetes has been for the individual to keep a paper logbook of their lifestyle. Another way is for an individual to simply rely on remembering facts about their lifestyle and then relay these details to their physician on each visit.

The aforementioned methods of recording lifestyle information are inherently difficult, time consuming, and possibly inaccurate. Paper logbooks are not necessarily always carried by an individual and may not be accurately completed when required. Such paper logbooks are small and it is therefore difficult to enter detailed information requiring detailed descriptors of lifestyle events. Furthermore, an individual may often forget key facts about their lifestyle when questioned by a physician who has to manually review and interpret information from a hand-written notebook. There is no analysis provided by the paper logbook to distill or separate the component information. Also, there are no graphical reductions or summary of the information. Entry of data into a secondary data storage system, such as a database or other electronic system, requires a laborious transcription of information, including lifestyle data, into this secondary data storage. Difficulty of data recordation encourages retrospective entry of pertinent information that results in inaccurate and incomplete records.

There currently exists a number of portable electronic devices that can measure glucose levels in an individual and store the levels for recalling or uploading to another computer for analysis. One such device is the Accu-Check™ Completer™ System from Roche Diagnostics, which provides limited functionality for storing lifestyle data. However, the Accu-Check™ Completer™ System only permits a limited selection of lifestyle variables to be stored in a meter. There is a no intelligent feedback from values previously entered into the meter and the user interface is unintuitive for an infrequent user of the meter.

### SUMMARY OF THE DISCLOSURE

In an embodiment, a diabetes management system is provided that includes a plurality of glucose test strips, a test strip port connector, and a diabetes data management unit. Each of the plurality of glucose test strips is configured to receive a physiological sample from a user. The test strip port connector is configured to receive the plurality of test strips. The diabetes data management device includes a housing, a microprocessor coupled to a memory, display, and power supply disposed proximate the housing. The microprocessor is coupled to the test strip sensor to provide data representative of a first group and second group of blood glucose values of the user over respective first and second time periods so that respective first and second medians of the first and second group are evaluated by the microprocessor to determine whether one of the first and second medians is significantly different enough to inform the user of the same on the display of the device.

In accordance with the embodiment, as set forth above, the first and second medians can be calculated by the microprocessor with glucose values including a common type of flag. The common type of flag can include at least one of a fasting flag or a bedtime flag.

In yet another embodiment, a method of detecting a fasting glucose concentration pattern is provided that includes obtaining a first group and second group of glucose measurements over a first time period and a second time period, respectively, via an analyte testing device; determining whether the fasting glucose concentrations of the first group is significantly different than the fasting glucose concentrations of the second group; calculating a first median and a second median of the glucose measurements over a first time period and a second time period, respectively; displaying a message indicating that the second group has a significantly higher fasting glucose concentration than the first group where the second median is greater than the first median, and the first group and second group are significantly different; and displaying a message indicating that the second group has a significantly lower fasting glucose concentration than the first group where the second median is less than the first median, and the first group and second group are significantly different.

In another embodiment, a method of detecting a fasting glucose concentration pattern for a day of the week is provided. The method includes obtaining a number of glucose measurements over a plurality of weeks, via an analyte testing device; determining whether the fasting glucose concentrations acquired on at least one day of the week is significantly different than the other days; displaying a message indicating that a particular day of the week has a significantly lower or significantly higher fasting glucose concentration than the other days of the week.

The significant difference may include a statistical difference. The statistical difference can be determined using a chi-squared test and the first group and the second group each has greater than ten fasting glucose concentrations.

The chi-squared value can be calculated using an equation,

$${χ}^{2} = {\sum }_{i = 1}^{n} \frac{{( {F}_{i} - {F}_{i , pre} )}^{2}}{{F}_{i \cdot pre}} + {\sum }_{i = 1}^{n} \frac{{( {F}_{i}^{′} - {F}_{i , pre}^{′} )}^{2}}{{F}_{i \cdot pre}^{′}} ,$$

where Fᵢ is an observed number of fasting glucose concentrations above an overall median during a time period i; F′ᵢ is an observed number of fasting glucose concentrations below or equal to an overall median during the time period i; F_{i,pre} is an expected number of fasting glucose concentrations above an overall median during the time period i; F′_{i,pre} is an expected number of fasting glucose concentrations below or equal to the overall median during the time period i; and n is a number of time periods

The method can further include determining that at least one of the time periods i is statistically different when the calculated chi-squared value is greater than a reference chi-squared value.

The method can further include calculating F_{i,pre} using an equation,

$${F}_{i , pre} = \frac{{\sum }_{i = 1}^{n} {F}_{i}}{{\sum }_{i = 1}^{n} {N}_{i}} * {N}_{i} ,$$

where Nᵢ represents a total number of flagged glucose measurements during a time period i.

The method can further include calculating F′_{i,pre} using an equation,

$${F}_{i , pre}^{′} = \frac{{\sum }_{i = 1}^{n} {F}_{i}^{′}}{{\sum }_{i = 1}^{n} {N}_{i}} * {N}_{i} ,$$

where Nᵢ represents a total number of flagged glucose measurements during a time period i.

In an embodiment, a method of detecting a bedtime glucose concentration pattern is provided that includes obtaining a first group and second group of glucose measurements over a first time period and a second time period, respectively, via an analyte testing device; determining whether the bedtime glucose concentrations of the first group is significantly different than the bedtime glucose concentrations of the second group; calculating a first median and a second median of the glucose measurements over a first time period and a second time period, respectively; displaying a message indicating that the second group has a significantly higher bedtime glucose concentration than the first group where the second median is greater than the first median, and the first group and second group are significantly different; and displaying a message indicating that the second group has a significantly lower bedtime glucose concentration than the first group where the second median is less than the first median, and the first group and second group are significantly different.

In another embodiment, a method of detecting a bedtime glucose concentration pattern for a day of the week is provided. The method includes obtaining a number of glucose measurements over a plurality of weeks, via an analyte testing device; determining whether the bedtime glucose concentrations acquired on at least one day of the week is significantly different than the other days; displaying a message indicating that a particular day of the week has a significantly lower or significantly higher bedtime glucose concentration than the other days of the week.

The significant difference includes a statistical difference. The statistical difference can be determined using a chi-squared test. In accordance with the embodiments, as set forth above the first group and the second group each have greater than ten bedtime glucose concentrations.

The chi-squared value can be calculated using an equation,

$${χ}^{2} = {\sum }_{i = 1}^{n} \frac{{( {B}_{i} - {B}_{i , pre} )}^{2}}{{B}_{i \cdot pre}} + {\sum }_{i = 1}^{n} \frac{{( {B}_{i}^{′} - {B}_{i , pre}^{′} )}^{2}}{{B}_{i \cdot pre}^{′}} ,$$

where Bᵢ is an observed number of bedtime glucose concentrations above an overall median during a time period i; B′ᵢ an observed number of bedtime glucose concentrations below or equal to an overall median during the time period i; B_{i,pre} is an expected number of bedtime glucose concentrations above an overall median during the time period i; B′_{i,pre} is an expected number of bedtime glucose concentrations below or equal to the overall median during the time period i; and n is a number of time periods

The method can further include determining that at least one of the time periods i is statistically different when the calculated chi-squared value is greater than a reference chi-squared value.

The method can further include calculating B_{i,pre} using an equation,

$${B}_{i , pre} = \frac{{\sum }_{i = 1}^{n} {B}_{i}}{{\sum }_{i = 1}^{n} {N}_{i}} * {N}_{i} ,$$

where Nᵢ represents a total number of flagged glucose measurements during a time period i.

The method can further include calculating B′_{i,pre} using an equation,

$${B}_{i , pre}^{′} = \frac{{\sum }_{i = 1}^{n} {B}_{i}^{′}}{{\sum }_{i = 1}^{n} {N}_{i}} * {N}_{i} ,$$

where Nᵢ represents a total number of flagged glucose measurements during a time period i.

These and other embodiments, features and advantages will become apparent to those skilled in the art when taken with reference to the following more detailed description of various exemplary embodiments of the invention in conjunction with the accompanying drawings that are first briefly described.

### BRIEF DESCRIPTION OF THE FIGURES

The accompanying drawings, which are incorporated herein and constitute part of this specification, illustrate presently preferred embodiments of the invention, and, together with the general description given above and the detailed description given below, serve to explain features of the invention (wherein like numerals represent like elements).

FIG. 1 illustrates a diabetes management system that includes an analyte measurement and management device and data communication devices.

FIG. 2A illustrates a top portion of a circuit board of the analyte measurement and management device.

FIG. 2B illustrates a bottom portion of the circuit board of the analyte measurement and management device.

FIG. 3 illustrates a schematic of the functional components of an insulin pump.

FIG. 4 illustrates a user interface of the analyte measurement and management device for detecting patterns in fasting glucose concentrations.

FIG. 5 is a flow chart illustrating a method of operating an analyte measurement device.

FIG. 6 is a flow chart illustrating a method of operating an analyte measurement device when only a single user interface button on the analyte measurement device is active.

FIG. 7 is a flow chart illustrating a method of operating an analyte measurement device where a user is queried when an analyte value is outside a predetermined range.

FIG. 8 is a flow chart illustrating a method of operating an analyte measurement device where a predetermined flag, an analyte value, and the date and time of a measurement are stored in the memory of the analyte measurement device.

FIG. 9 is a flow chart illustrating a method of operating an analyte measurement device after inserting a test strip into a strip port in the analyte measurement device.

FIG. 10 is a flow chart illustrating a method of operating an analyte measurement device after inserting a test strip into a strip port in the analyte measurement device and either entering or confirming calibration parameters of the test strip.

FIG. 11 is a flow chart illustrating a method of operating an analyte measurement device after inserting a test strip into a strip port in the analyte measurement device thereby turning the analyte measurement device on.

FIG. 12 is a flow chart illustrating an alternative method of operating an analyte measurement device where all but one user interface buttons are ignored.

FIG. 13 is a flow chart illustrating a method of operating an analyte measurement device and actions taken by the analyte measurement device.

FIG. 14 illustrates a series of user interface screens used in a method of operating an analyte measurement device.

FIG. 15 illustrates various navigation paths for the selection of various predetermined flags.

FIGS. 16A-16D illustrate various user interface screens that can be used to display respective warning messages instead of a numerical value for a blood glucose measurement along with a flag that can be associated with the warning message according to an exemplary embodiment described and illustrated herein.

FIGS. 17A-17I illustrate various user interface screens to provide additional statistical information regarding blood glucose measurements.

FIG. 18 illustrates a flow chart of a method of detecting a significant change in fasting glucose concentrations for two reporting periods.

FIG. 19 illustrates a chi-squared table that can be used to determine a statistically significant pattern based on a patient's fasting glucose concentration.

FIG. 20 illustrates a flow chart of a method of detecting a significant change in fasting glucose concentrations for a day of the week.

FIG. 21 illustrates a flow chart of a method of detecting a significant change in bedtime glucose concentrations for two reporting periods.

FIG. 22 illustrates a chi-squared table that can be used to determine a statistically significant pattern based on a patient's bedtime glucose concentration.

FIG. 23 illustrates a flow chart representative of a method of detecting a significant change in bedtime glucose concentrations for a day of the week.

FIG. 24 illustrates an output on a report where there was a significant change in bedtime glucose concentrations for two reporting periods.

FIG. 25 illustrates an output on a report where there was a significant change in bedtime glucose concentrations for a day of the week.

### DETAILED DESCRIPTION OF THE EXEMPLARY FIGURES

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient,” “host,” “user,” and “subject” refer to any human or animal subject and are not intended to limit the systems or methods to human use, although use of the subject invention in a human patient represents a preferred embodiment.

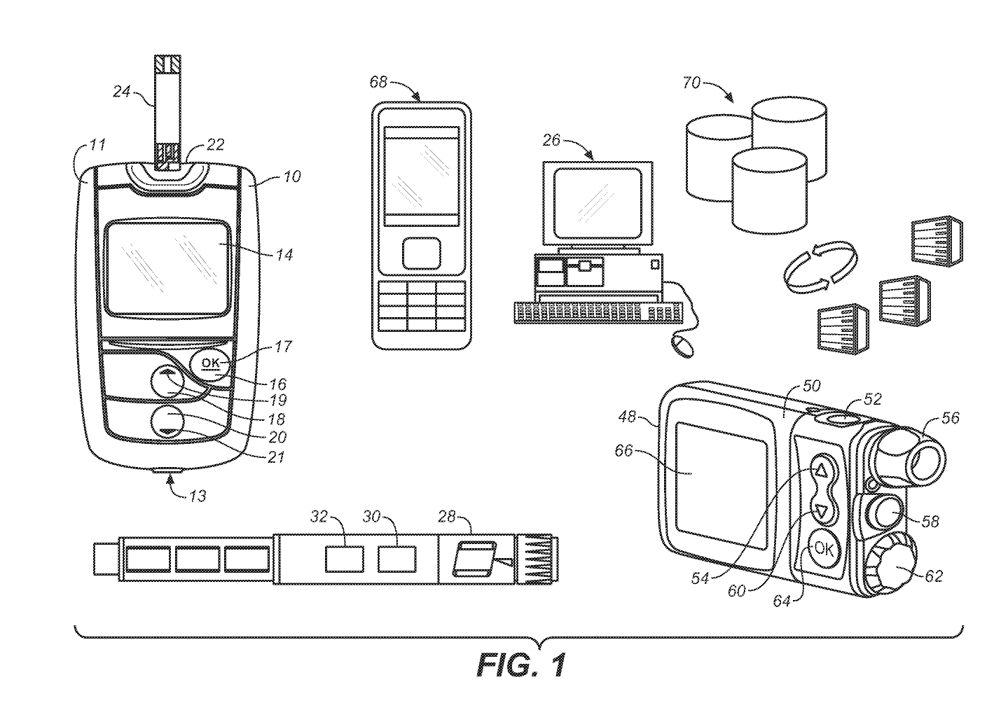

FIG. 1 illustrates a diabetes management system that includes an analyte measurement and management device 10, therapeutic dosing devices (28 or 48), and data/communication devices (68, 26, or 70). Analyte measurement and management device 10 can be configured to wirelessly communicate with a handheld glucose-insulin data management unit or DMU such as, for example, an insulin pen 28, an insulin pump 48, a mobile phone 68, or through a combination of the exemplary handheld glucose-insulin data management unit devices in communication with a personal computer 26 or network server 70, as described herein. As used herein, the nomenclature “DMU” represents either individual unit 10, 28, 48, 68, separately or all of the handheld glucose-insulin data management units (28, 48, 68) usable together in a disease management system. Further, the analyte measurement and management device or DMU 10 is intended to include a glucose meter, a meter, an analyte measurement device, an insulin delivery device or a combination of or an analyte testing and drug delivery device. In an embodiment, analyte measurement and management device 10 may be connected to personal computer 26 with a cable. In an alternative, the DMU may be connected to the computer 26 or server 70 via a suitable wireless technology such as, for example, GSM, CDMA, BlueTooth, WiFi and the like.

Glucose meter 10 can include a housing 11, user interface buttons (16, 18, and 20), a display 14, a strip port connector 22, and a data port 13, as illustrated in FIG. 1. User interface buttons (16, 18, and 20) can be configured to allow the entry of data, navigation of menus, and execution of commands. Data can include values representative of analyte concentration, and/or information, which are related to the everyday lifestyle of an individual. Information, which is related to the everyday lifestyle, can include food intake, medication use, occurrence of health check-ups, and general health condition and exercise levels of an individual. Specifically, user interface buttons (16, 18, and 20) include a first user interface button 16, a second user interface button 18, and a third user interface button 20. User interface buttons (16, 18, and 20) include a first marking 17, a second marking 19, and a third marking 21, respectively, which allow a user to navigate through the user interface.

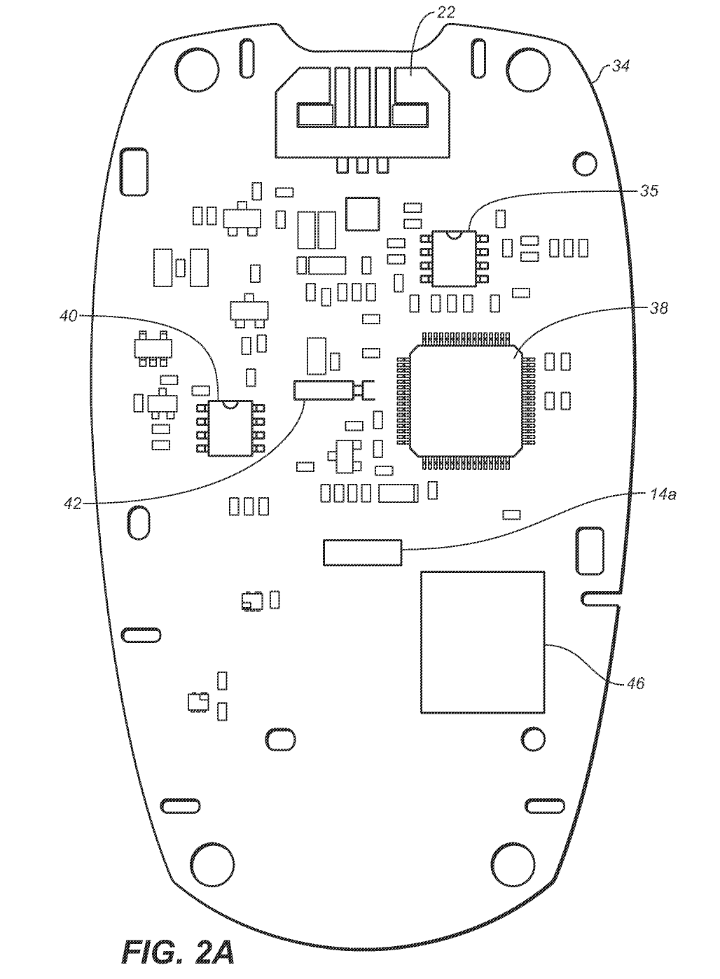

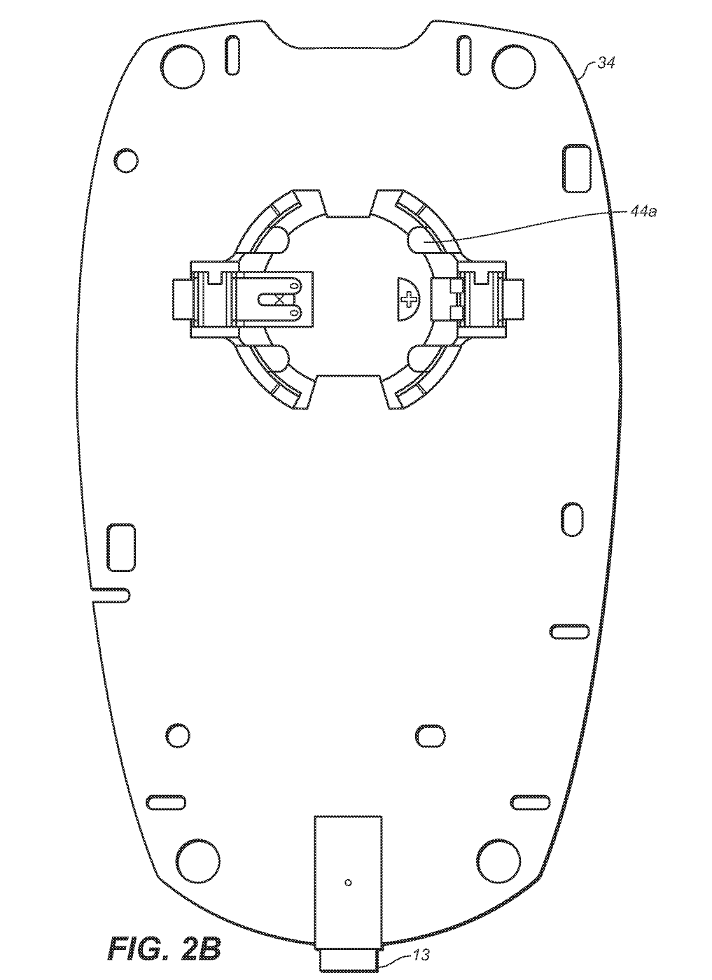

The electronic components of meter 10 can be disposed on a circuit board 34 that is within housing 11. FIGS. 2A and 2B illustrate the electronic components disposed on a top surface and a bottom surface of circuit board 34, respectively. On the top surface, the electronic components include a strip port connector 22, an operational amplifier circuit 35, a microcontroller 38, a display connector 14a, a non-volatile memory 40, a clock 42, and a first wireless module 46. On the bottom surface, the electronic components include a battery connector 44a and a data port 13. Microcontroller 38 can be electrically connected to strip port connector 22, operational amplifier circuit 35, first wireless module 46, display 14, non-volatile memory 40, clock 42, battery connector 44a, data port 13, and user interface buttons (16, 18, and 20).

Operational amplifier circuit 35 can include two or more operational amplifiers configured to provide a portion of the potentiostat function and the current measurement function. The potentiostat function can refer to the application of a test voltage between at least two electrodes of a test strip. The current function can refer to the measurement of a test current resulting from the applied test voltage. The current measurement may be performed with a current-to-voltage converter. Microcontroller 38 can be in the form of a mixed signal microprocessor (MSP) such as, for example, the Texas Instrument MSP 430. The MSP 430 can be configured to also perform a portion of the potentiostat function and the current measurement function. In addition, the MSP 430 can also include volatile and non-volatile memory. In another embodiment, many of the electronic components can be integrated with the microcontroller in the form of an application specific integrated circuit (ASIC).

Strip port connector 22 can be configured to form an electrical connection to the test strip. Display connector 14a can be configured to attach to display 14. Display 14 can be in the form of a liquid crystal display for reporting measured glucose levels, and for facilitating entry of lifestyle related information. Display 14 can optionally include a backlight. Data port 13 can accept a suitable connector attached to a connecting lead, thereby allowing glucose meter 10 to be linked to an external device such as a personal computer. Data port 13 can be any port that allows for transmission of data such as, for example, a serial, USB, or a parallel port. Clock 42 can be configured for measuring time and be in the form of an oscillating crystal. Battery connector 44a can be configured to be electrically connected to a power supply.

In one exemplary embodiment, test strip 24 can be in the form of an electrochemical glucose test strip. Test strip 24 can include one or more working electrodes and a counter electrode. Test strip 24 can also include a plurality of electrical contact pads, where each electrode can be in electrical communication with at least one electrical contact pad. Strip port connector 22 can be configured to electrically interface to the electrical contact pads and form electrical communication with the electrodes. Test strip 24 can include a reagent layer that is disposed over at least one electrode. The reagent layer can include an enzyme and a mediator. Exemplary enzymes suitable for use in the reagent layer include glucose oxidase, glucose dehydrogenase (with pyrroloquinoline quinone co-factor, “PQQ”), and glucose dehydrogenase (with flavin adenine dinucleotide co-factor, “FAD”). An exemplary mediator suitable for use in the reagent layer includes ferricyanide, which in this case is in the oxidized form. The reagent layer can be configured to physically transform glucose into an enzymatic by-product and in the process generate an amount of reduced mediator (e.g., ferrocyanide) that is proportional to the glucose concentration. The working electrode can then measure a concentration of the reduced mediator in the form of a current. In turn, glucose meter 10 can convert the current magnitude into a glucose concentration.

Referring back to FIG. 1, insulin pen 28 can include a housing, preferably elongated and of sufficient size to be handled by a human hand comfortably. The device 28 can be provided with an electronic module 30 to record dosage amounts delivered by the user. The device 28 may include a second wireless module 32 disposed in the housing that, automatically without prompting from a user, transmits a signal to first wireless module 46 of the DMU 10. The wireless signal can include, in an exemplary embodiment, data to (a) type of therapeutic agent delivered; (b) amount of therapeutic agent delivered to the user; or (c) time and date of therapeutic agent delivery.

In one embodiment, a therapeutic delivery device can be in the form of a “user-activated” therapeutic delivery device, which requires a manual interaction between the device and a user (for example, by a user pushing a button on the device) to initiate a single therapeutic agent delivery event and that in the absence of such manual interaction deliver no therapeutic agent to the user. A non-limiting example of such a user-activated therapeutic agent delivery device is described in co-pending U.S. Non-Provisional application Ser. No. 12/407,173 (tentatively identified by Attorney Docket No. LFS-5180USNP); 12/417,875 (tentatively identified by Attorney Docket No. LFS-5183USNP); and 12/540,217 (tentatively identified by Attorney Docket No. DDI-5176USNP), which is hereby incorporated in whole by reference with a copy attached hereto this application. Another non-limiting example of such a user-activated therapeutic agent delivery device is an insulin pen 28. Insulin pens can be loaded with a vial or cartridge of insulin, and can be attached to a disposable needle. Portions of the insulin pen can be reusable, or the insulin pen can be completely disposable. Insulin pens are commercially available from companies such as Novo Nordisk, Aventis, and Eli Lilly, and can be used with a variety of insulin, such as Novolog, Humalog, Levemir, and Lantus.

Referring to FIG. 1, a therapeutic dosing device can also be a pump 48 that includes a housing 50, a backlight button 52, an up button 54, a cartridge cap 56, a bolus button 58, a down button 60, a battery cap 62, an OK button 64, and a display 66. Pump 48 can be configured to dispense medication such as, for example, insulin for regulating glucose levels.

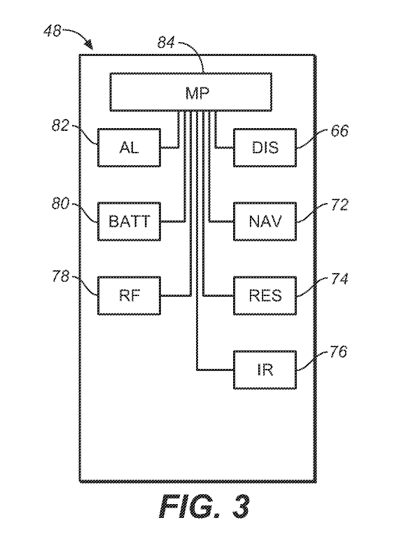

Referring to FIG. 3, pump 48 includes the following functional components that are a display (DIS) 66, navigational buttons (NAV) 72, a reservoir (RES) 74, an infrared communication port (IR) 76, a radio frequency module (RF) 78, a battery (BAT) 80, an alarm module (AL) 82, and a microprocessor (MP) 84. Note that navigational buttons 72 can include up button 54, down button 60, and ok button 64.

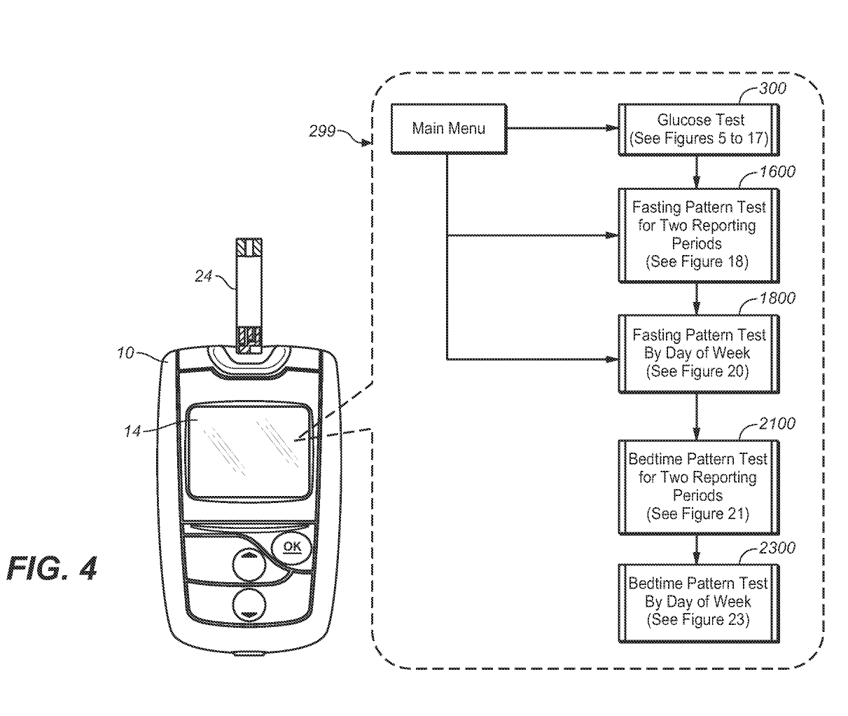

FIG. 4 illustrates a user interface 299 that is programmed for a particular device, such as, for example, glucose meter, pump, pen, or mobile hand-held computing device. The programmed user interface 299 provides pattern recognition for fasting and bedtime glucose concentrations. In an embodiment, programs and methods for conducting user interface 299 can be stored on non-volatile memory 40 of glucose meter 10. A microprocessor can be programmed to generally carry out the steps of user interface 299. The microprocessor can be part of a particular device, such as, for example, a glucose meter, an insulin pen, an insulin pump, a server, a mobile phone, personal computer, or mobile hand held device. Steps and instructions of user interface 299 can be displayed on display 14 of glucose meter 10. Significant increases or decreases in fasting glucose concentrations can be detected so that warning messages can be outputted via a display of the DMU or the glucose meter to a user. Note that a warning message may be annunciated. As used here, the term “annunciated” and variations on the root term indicate that an announcement may be provided via text, audio, visual or a combination of all modes of communication to a user, a caretaker of the user, or a healthcare provider.

In another embodiment, the software for user interface 299 can stored on the memory of computer 26, cell phone 68, or server 70. Glucose measurements, date and time, and fasting flag information can be transferred to the DMU through a wired or wireless manner and then processed using user interface 299.

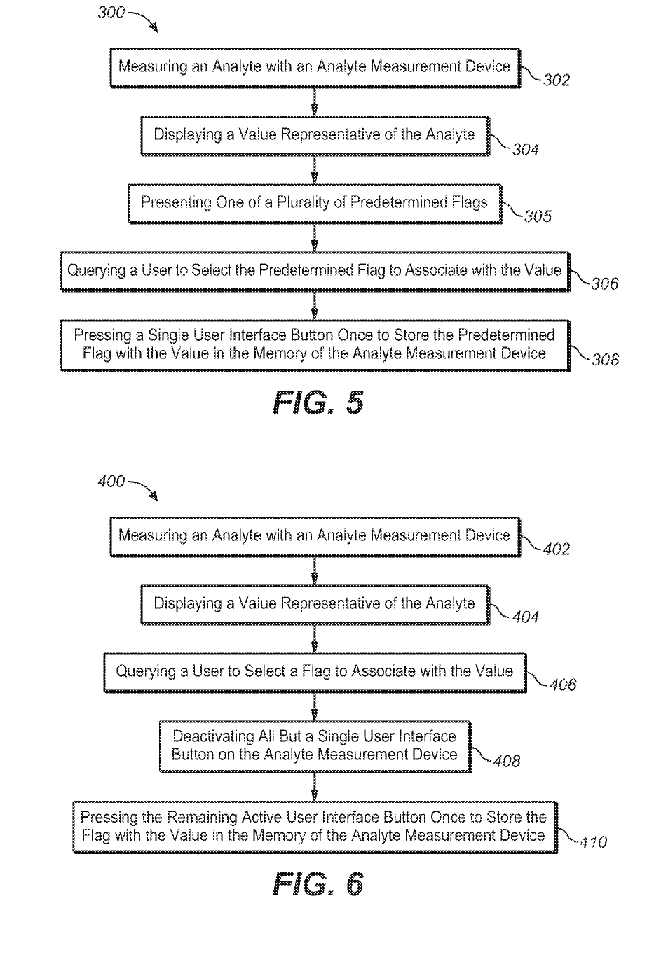

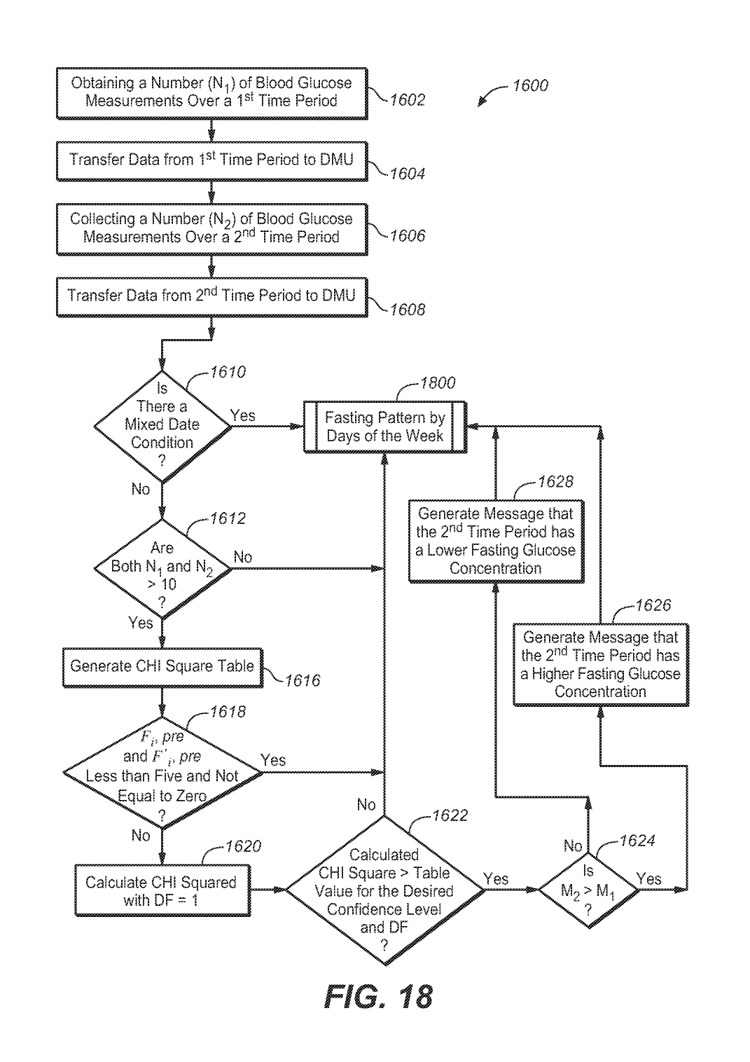

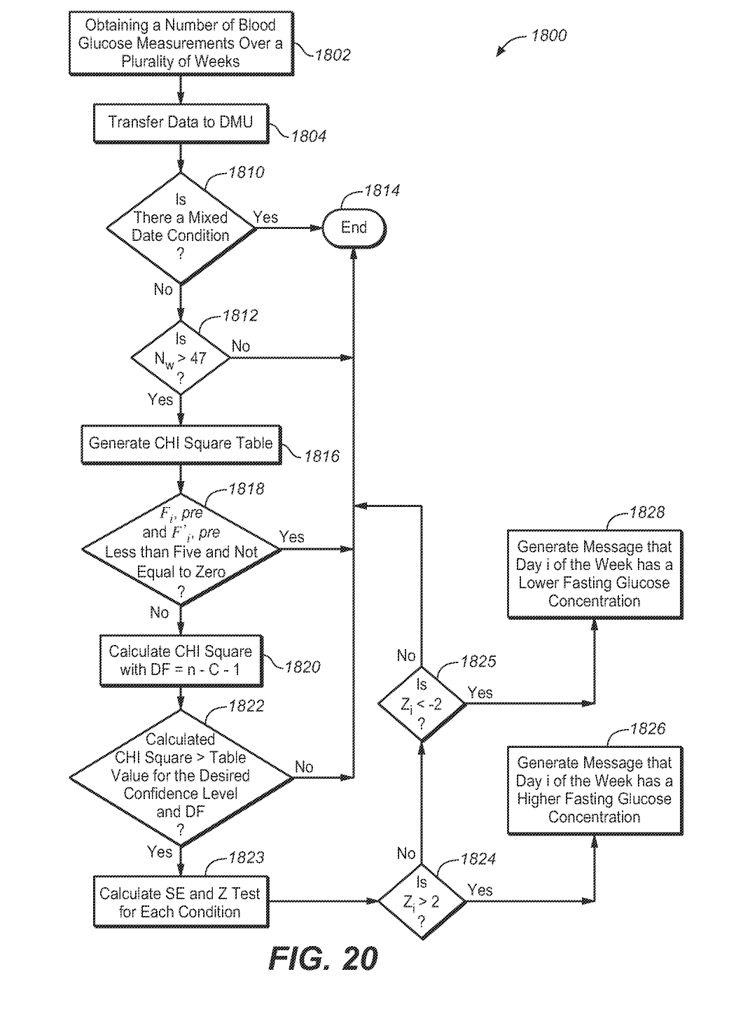

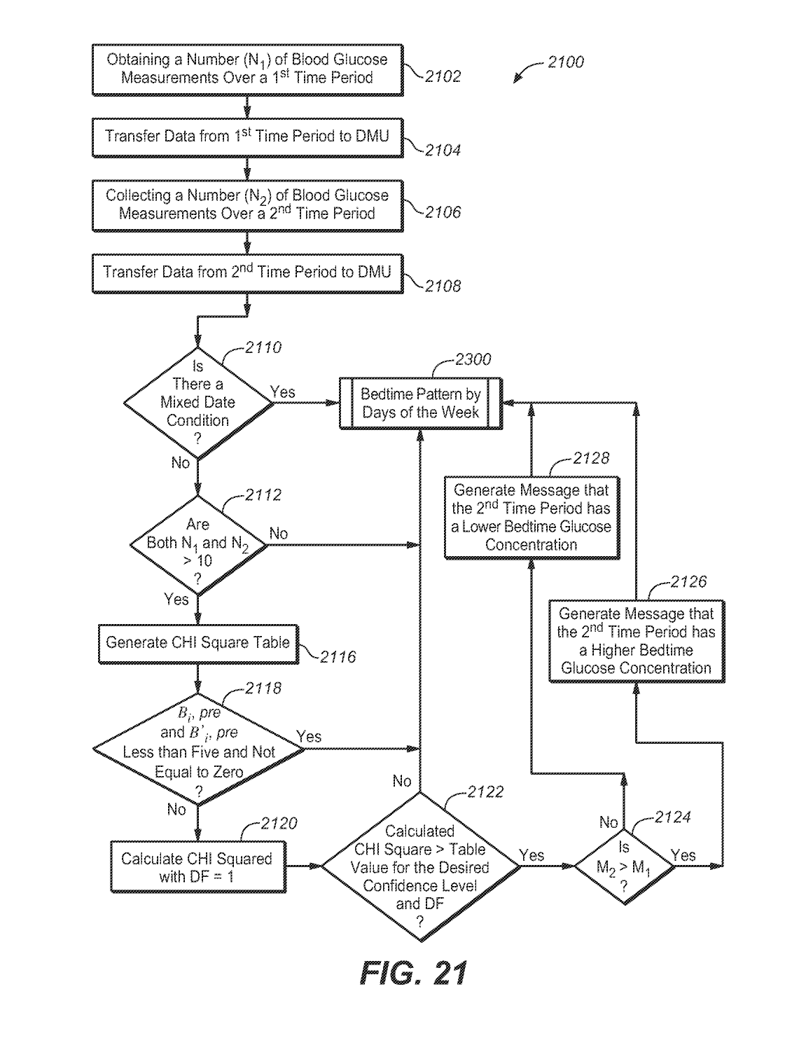

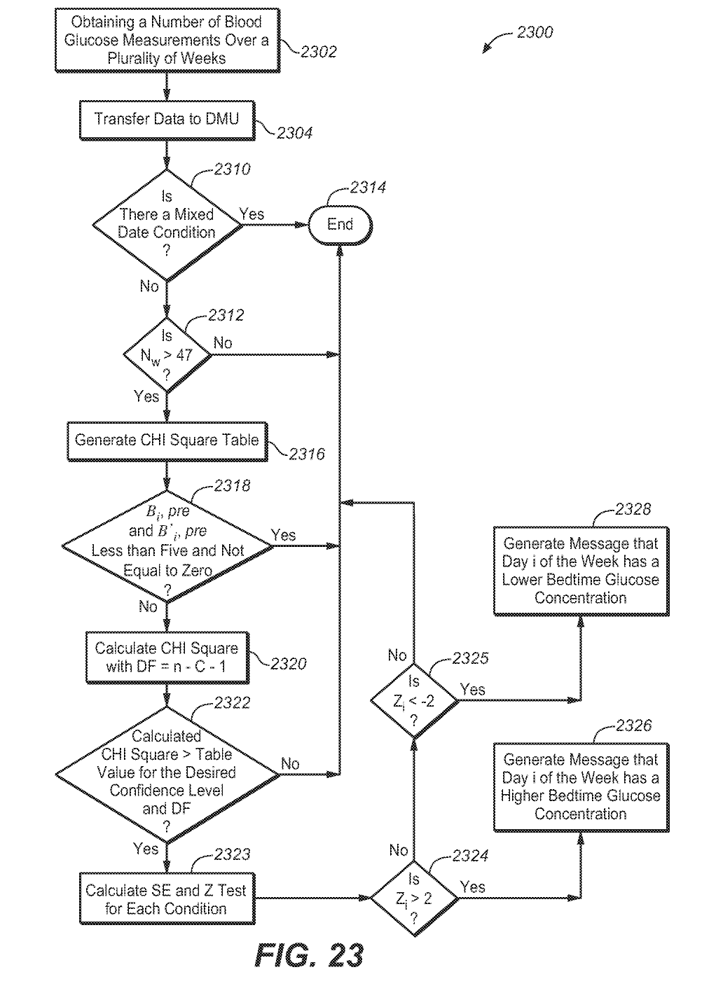

From main menu 299, a user can opt to perform a glucose test 300 along with suitable flags, prompts, or messages for such test (see FIGS. 5 to 17) or a fasting pattern test for two reporting periods 1600 (see FIG. 18), by the day of the week 1800 (see FIG. 20), a bedtime pattern test for two reporting periods 2100 (see FIG. 21), by the day of the week 2300 (see FIG. 23), as shown in FIG. 4. Glucose test 300 can include the measurement of glucose with a test strip and the flagging of the measurement. In an embodiment, a user can flag the measurement as fasting where the user has not recently consumed food. The following FIGS. 5 to 17 will describe various methods of performing a glucose test that includes a flagging of the measurement with a particular type of flag such as, for example, a fasting flag.

FIG. 5 is an exemplary flow chart illustrating a method 300 of operating an analyte measurement device. A microprocessor can be programmed to generally carry out the steps of method 300. The microprocessor can be part of a particular device, such as, for example, a glucose meter, an insulin pen, an insulin pump, a server, a mobile phone, personal computer, or mobile hand held device. Method 300 includes steps 302, 304, 305, 306, and 308. In step 302, an analyte measuring device measures an analyte. In step 304, the analyte measuring device displays a value representative of the analyte. In step 305, the analyte measuring device presents one of a plurality of predetermined flags. In step 306, the analyte measuring device queries the user to select a predetermined flag to associate with the displayed value. In step 308, a single user interface button is pressed once, causing the predetermined flag and the displayed value to be stored in the memory of the analyte measurement device. Preferably, the analyte measurement device may include a display, a user interface, a processor, and a memory and user interface buttons. Similarly, querying may include repetitively flashing on the display an icon representative of one of the user interface buttons to prompt a selection of such user interface button. Preferably, the icon may be selected from a group consisting of a first triangle and a second triangle having a smaller area than the first triangle.

FIG. 6 is an exemplary flow chart illustrating a method 400 of operating an analyte measurement device when only a single user interface button on the analyte measurement device is active, i.e., the remaining interface buttons are not active. A microprocessor can be programmed to generally carry out the steps of method 400. The microprocessor can be part of a particular device, such as, for example, a glucose meter, an insulin pen, an insulin pump, a server, a mobile phone, personal computer, or mobile hand held device. Method 400 includes steps 402, 404, 406, 408, and 410. In step 402, an analyte measuring device measures an analyte. In step 404, the analyte measuring device displays a value representative of the analyte. In step 406, the analyte measuring device queries the user to select a flag to associate with the displayed value. In step 408, the analyte measuring device deactivates all but a single user interface button. In step 410, the active user interface button is pressed once, causing the flag and the displayed value to be stored in the memory of the analyte measurement device. Preferably, user interface buttons may include an “up” button, a “down” button, and an “enter” or “OK” button. Preferably, user selectable flags may include a before meal flag, an after meal flag, a fasting flag, bedtime, or a blank flag. Preferably, queries may be used whenever a measuring step has been completed.

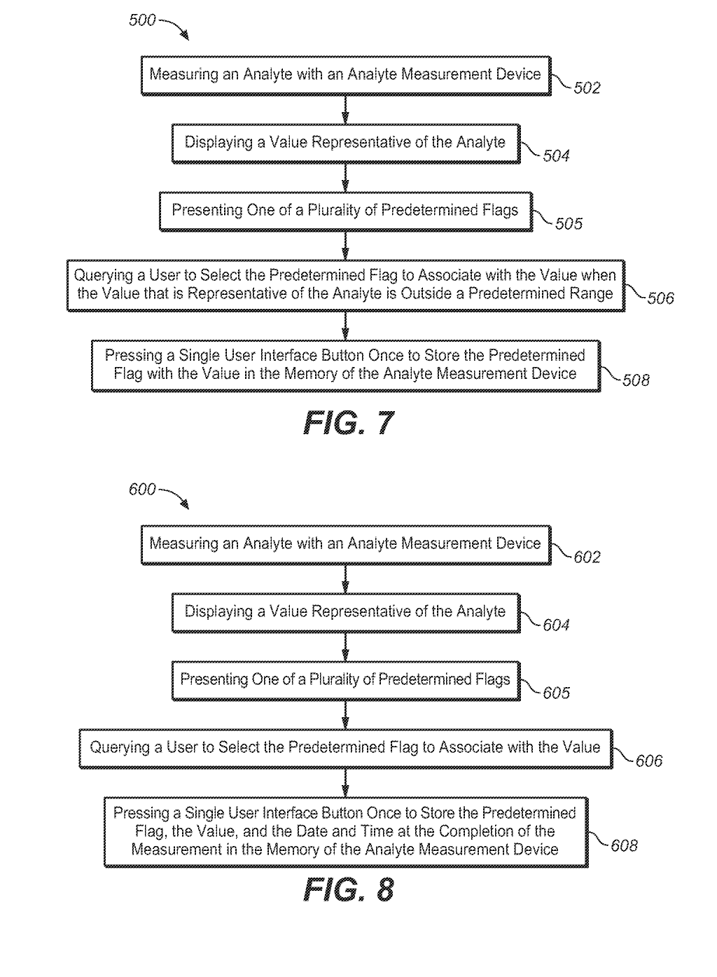

FIG. 7 is an exemplary flow chart illustrating a method 500 of operating an analyte measurement device where a user is queried when an analyte value is outside a predetermined range. A microprocessor can be programmed to generally carry out the steps of method 500. The microprocessor can be part of a particular device, such as, for example, a glucose meter, an insulin pen, an insulin pump, a server, a mobile phone, personal computer, or mobile hand held device. Method 500 includes steps 502, 504, 505, 506, and 508. In step 502, an analyte measuring device measures an analyte. In step 504, the analyte measuring device displays a value representative of the analyte. In step 505, the analyte measuring device presents one of a plurality of predetermined flags. In step 506, the analyte measuring device queries the user to select a predetermined flag to associate with the displayed value when the displayed value is outside a predetermined range. In step 508, a single user interface button is pressed once, causing the predetermined flag and the displayed value to be stored in the memory of the analyte measurement device.

FIG. 8 is an exemplary flow chart illustrating a method 600 of operating an analyte measurement device where a predetermined flag, an analyte value, and the date and time of a measurement are stored in the memory of the analyte measurement device. A microprocessor can be programmed to generally carry out the steps of method 600. The microprocessor can be part of a particular device, such as, for example, a glucose meter, an insulin pen, an insulin pump, a server, a mobile phone, personal computer, or mobile hand held device. Method 600 includes steps 602, 604, 605, 606, and 608. In step 602, an analyte measuring device measures an analyte. In step 604, the analyte measuring device displays a value representative of the analyte. In step 605, the analyte measuring device presents one of a plurality of predetermined flags. In step 606, the analyte measuring device queries the user to select a predetermined flag to associate with the displayed value. In step 608, a single user interface button is pressed once, causing the predetermined flag, the displayed value, and the date and time at the completion of the measurement to be stored in the memory of the analyte measurement device. Preferably, the analyte measuring device may include a glucose meter.

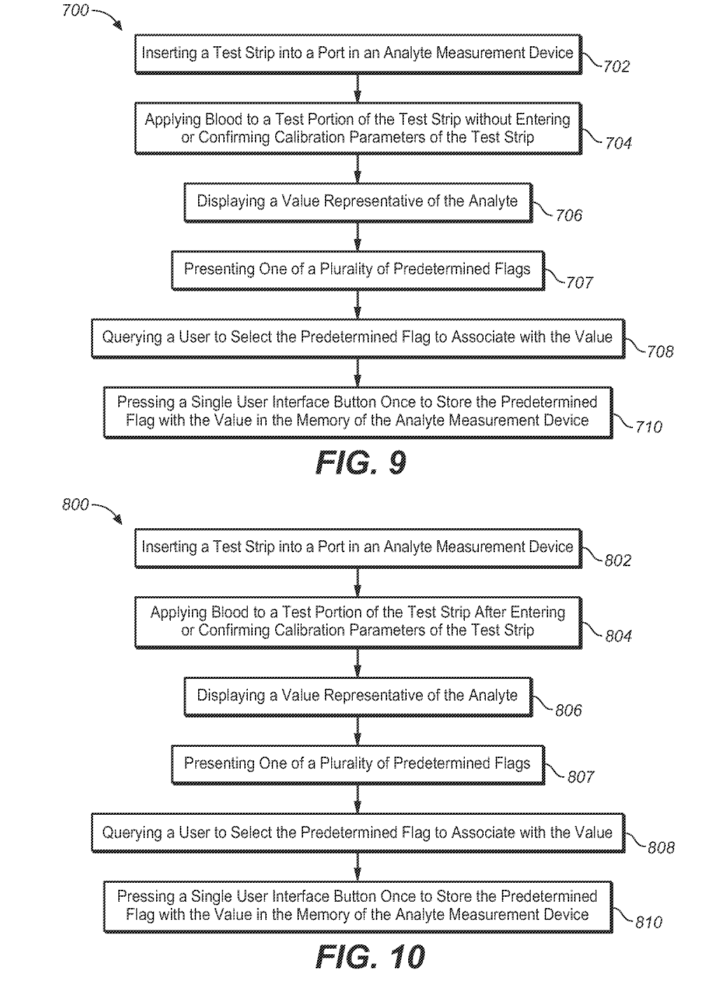

FIG. 9 is an exemplary flow chart illustrating a method 700 of operating an analyte measurement device after inserting a test strip 10 into a strip port 113 in the analyte measurement device. A microprocessor can be programmed to generally carry out the steps of method 700. The microprocessor can be part of a particular device, such as, for example, a glucose meter, an insulin pen, an insulin pump, a server, a mobile phone, personal computer, or mobile hand held device. Method 700 includes steps 702, 704, 706, 707, 708, and 710. In step 702, a test strip 10 is inserted into a strip port in an analyte measurement device. In step 704, blood is applied to a test portion (the portion distal from the strip port 112) of the test strip 10 without entering or confirming calibration parameters of the test strip 10. In step 706, the analyte measuring device displays a value representative of the analyte. In step 707, the analyte measuring device presents one of a plurality of predetermined flags. In step 708, the analyte measuring device queries the user to select a predetermined flag to associate with the displayed value. In step 710, a single user interface button is pressed once, causing the predetermined flag and the displayed value to be stored in the memory of the analyte measurement device. Preferably, measuring may include: inserting a test strip 10 into a strip port in the analyte measurement device, then depositing a sample of blood on a testing portion of the test strip 10 without entering a calibration parameter for the test strip 10.

FIG. 10 is an exemplary flow chart illustrating a method 800 of operating an analyte measurement device after inserting a test strip 10 into a strip port in the analyte measurement device and either entering or confirming calibration parameters of the test strip 10. A microprocessor can be programmed to generally carry out the steps of method 800. The microprocessor can be part of a particular device, such as, for example, a glucose meter, an insulin pen, an insulin pump, a server, a mobile phone, personal computer, or mobile hand held device. Method 800 includes steps 802, 804, 806, 807, 808, and 810. In step 802, a test strip 10 is inserted into a strip port in an analyte measurement device. In step 804, blood is applied to a test portion of the test strip 10 after entering or confirming calibration parameters of the test strip 10. In step 806, the analyte measuring device displays a value representative of the analyte. In step 807, the analyte measuring device presents one of a plurality of predetermined flags. In step 808, the analyte measuring device queries the user to select a predetermined flag to associate with the displayed value. In step 810, a single user interface button is pressed once, causing the predetermined flag and the displayed value to be stored in the memory of the analyte measurement device. Preferably, the measuring may include: inserting a test strip 10 into a strip port in the measurement device; inputting a calibration parameter for the test strip 10 via the user interface buttons of the device; and depositing a blood sample on a testing portion of the test strip 10.

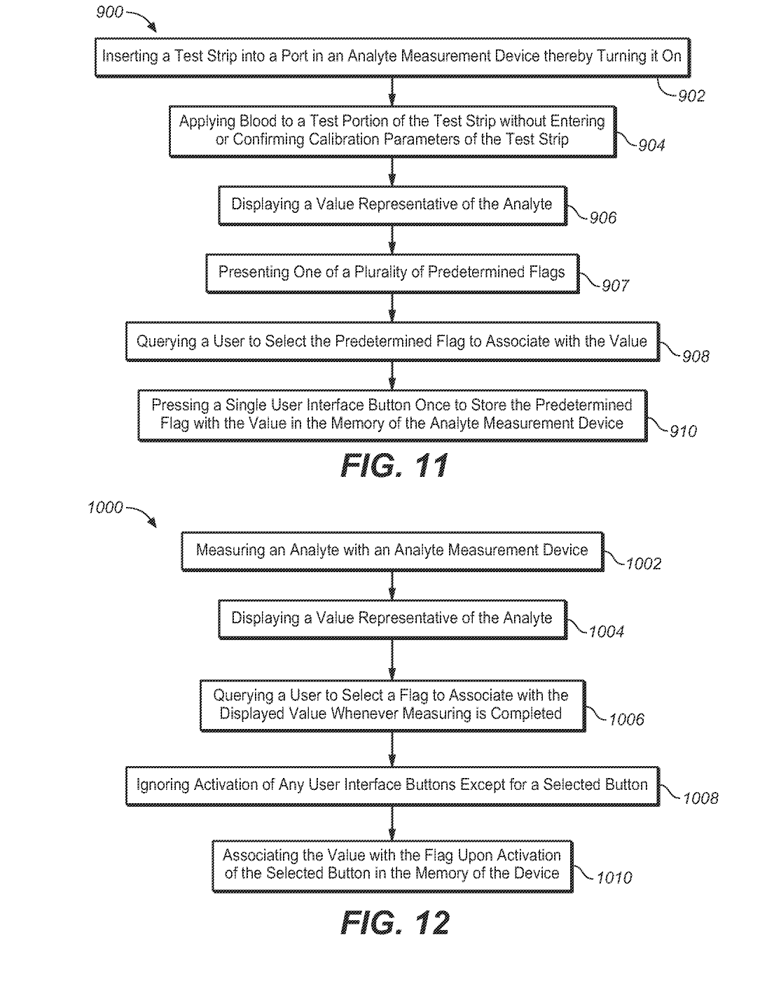

FIG. 11 is an exemplary flow chart illustrating a method 900 of operating an analyte measurement device after inserting a test strip 10 into a strip port in the analyte measurement device thereby turning the analyte measurement device on. A microprocessor can be programmed to generally carry out the steps of method 900. The microprocessor can be part of a particular device, such as, for example, a glucose meter, an insulin pen, an insulin pump, a server, a mobile phone, personal computer, or mobile hand held device. Method 900 includes steps 902, 904, 906, 907, 908, and 910. In step 902, a test strip 10 is inserted into a strip port in an analyte measurement device, thereby turning it on. In step 904, blood is applied to a test portion of the test strip 10 without entering or confirming calibration parameters of the test strip 10. In step 906, the analyte measuring device displays a value representative of the analyte. In step 907, the analyte measuring device presents one of a plurality of predetermined flags. In step 908, the analyte measuring device queries the user to select a predetermined flag to associate with the displayed value. In step 910, a single user interface button is pressed once, causing the predetermined flag and the displayed value to be stored in the memory of the analyte measurement device. Preferably, the inserting may include turning on the measurement device when the strip is fully inserted into the strip port. Preferably, one of a plurality of user selectable predetermined flags may be selected from a group consisting essentially of at least one of a comment title, a plurality of comments, comment page number, no comment, not enough food, too much food, mild exercise, strenuous exercise, medication, stress, illness, hypoglycemic state, menses, vacation, and combinations thereof. Preferably, a plurality of menus may be displayed. Preferably, one of a plurality of menus may include a prompt for last result, all results, result average, and set up. Preferably, a plurality of menus may include a display of a prompt for all results average, before meal average, after meal average.

In an alternative embodiment, certain keys on the meter can be disabled or ignored to ensure simplicity in the operation of the device. For example, in FIG. 12, all but one user interface buttons are ignored in method 1000. A microprocessor can be programmed to generally carry out the steps of method 1000. The microprocessor can be part of a particular device, such as, for example, a glucose meter, an insulin pen, an insulin pump, a server, a mobile phone, personal computer, or mobile hand held device. Method 1000 includes steps 1002, 1004, 1006, 1008, and 1010. In step 1002, an analyte measuring device measures an analyte. In step 1004, the analyte measuring device displays a value representative of the analyte. In step 1006, the analyte-measuring device queries the user to select a flag to associate with the displayed value whenever measuring is completed. In step 1008, the analyte measuring device ignores activation of all but a single user interface button. In step 1010, the single active user interface button is pressed once, causing the flag and the displayed value to be stored in the memory of the analyte measurement device. In an embodiment, the analyte measurement device may turn off without storing a flag if the user does not press the user interface button after a pre-determined period of time.

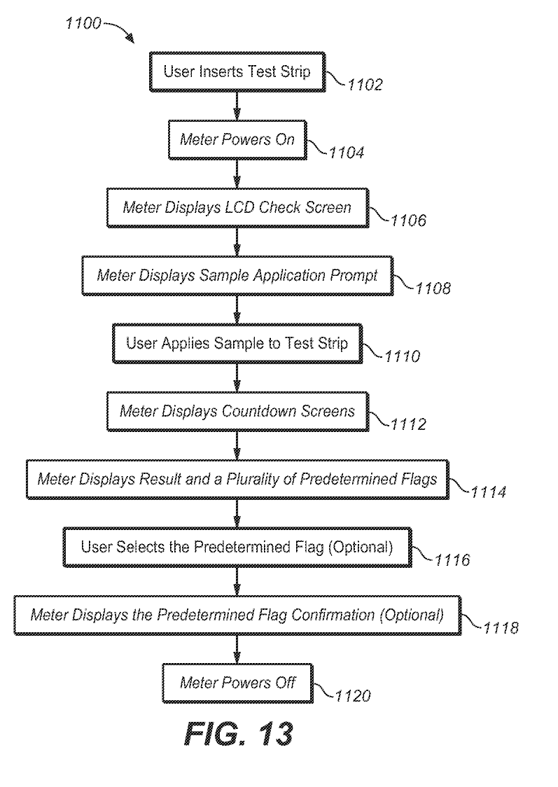

FIG. 13 is an exemplary flow chart illustrating a method 1100 of operating an analyte measurement device and actions taken by the analyte measurement device. A microprocessor can be programmed to generally carry out the steps of method 1100. The microprocessor can be part of a particular device, such as, for example, a glucose meter, an insulin pen, an insulin pump, a server, a mobile phone, personal computer, or mobile hand held device. Method 1100 includes steps 1102, 1104, 1106, 1108, 1110, 1112, 1114, 1116, 1118, and 1120. In step 1102, a user inserts a test strip 10 into a strip port in an analyte measurement device. In step 1104, the analyte measuring device turns on due to insertion of the test strip 10. In step 1106, the analyte-measuring device displays an LCD check screen. In step 1108, the analyte measuring device displays a sample application prompt. In step 1110, the user applies sample to the test strip 10. In step 1112, the analyte measuring device displays a series of countdown screens. In step 1114, the analyte measuring device displays a value representative of the analyte and queries the user to select one of a plurality of predetermined flags to associate with the displayed value. In step 1116, the user selects a predetermined flag, causing the predetermined flag and the displayed value to be stored in the memory of the analyte measurement device. In step 1118, the analyte measurement device displays a predetermined flag confirmation. In step 1120, the analyte measurement device turns off after a predetermined time, without interaction from the user.

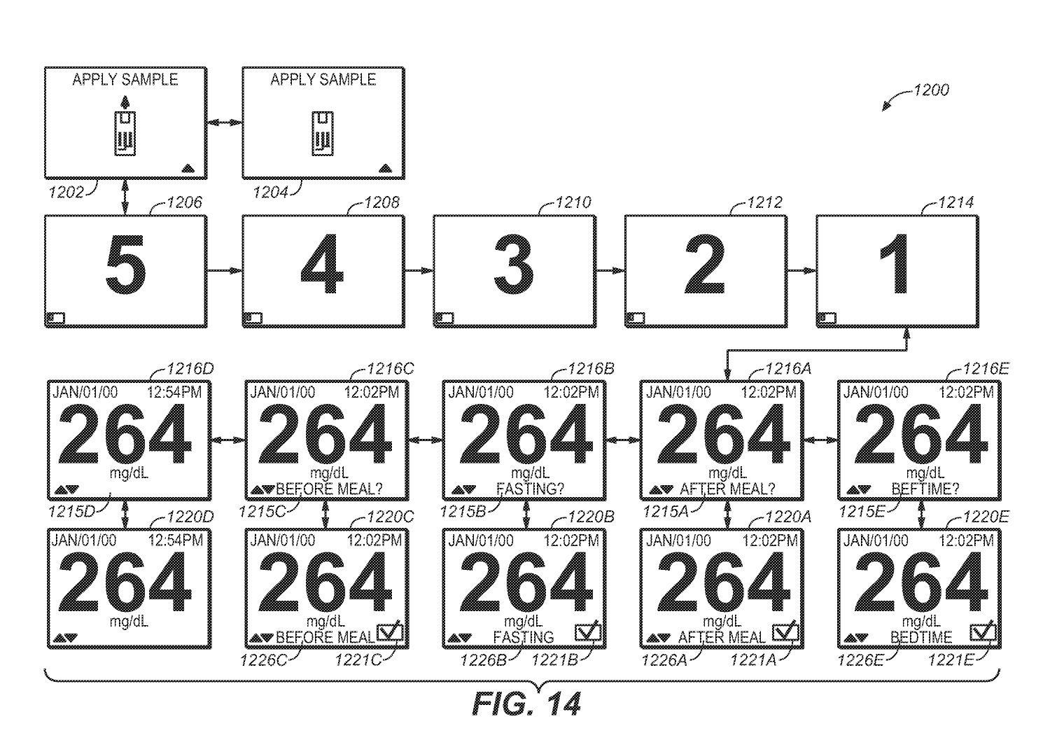

FIG. 14 illustrates a series of user interface screens displayed during a method 1200 of operating an analyte measurement device. Method 1200 includes screens 1202, 1204, 1206, 1208, 1210, 1212, 1214, 1216A, 1216B, 1216C, 1216D, 1216E,1220A, 1220B, 1220C, 1220D, and 1220E. In screens 1202 and 1204, the user is prompted to apply a physiological sample to a test strip 10 that has been inserted into a strip port in an analyte measurement device. In screen 1202 an icon symbolizing a drop of blood is displayed, while in screen 1204 there is no icon symbolizing a drop of blood. Screens 1202 and 1204 are alternated, creating the impression of a blinking drop of blood. Once sample is applied to the test strip 10, screens 1206, 1208, 1210, 1212, and 1214 are displayed, in succession. Screens 1206 through 1214 provide a countdown to result that is approximately 5 seconds in duration. In screens 1216A through 1216E, the analyte measuring device displays a value representative of the analyte and queries the user to select one of a plurality of predetermined flags to associate with the displayed value. A user can alternate between screens 1216A through 1216E by pressing a user interface button, such as the up button or the down button. Screen 1216A includes after meal flag 1215A, screen 1216B includes fasting flag 1215B, screen 1216C includes before meal flag 1215C, screen 1216E includes bedtime flag 1215E, and screen 1216D includes blank flag 1215D. Any one of flags 1215A through 1215E can be selected by pressing a user interface button (such as, for example, an “OK” button) while the flag is displayed. Once a flag is selected, one of screens 1220A through 1220E is displayed. Screen 1220A is displayed when an after meal flag 1215A is selected, screen 1220B is displayed when a fasting flag 1215B is selected, screen 1220C is displayed when a before meal flag 1215C is selected, screen 1220E is displayed when a bedtime flag 1215E is selected, and screen 1220D is displayed when a blank flag 1215D is selected. Screens 1220A, 1220B, 1220C, and 1220E include confirmation icons 1221A, 1220B, 1221C, and 1220E indicating that the corresponding flag has been selected. Similarly, the querying may include repetitively flashing on the display an icon representative of a single user interface button to prompt selection of the single user interface button.

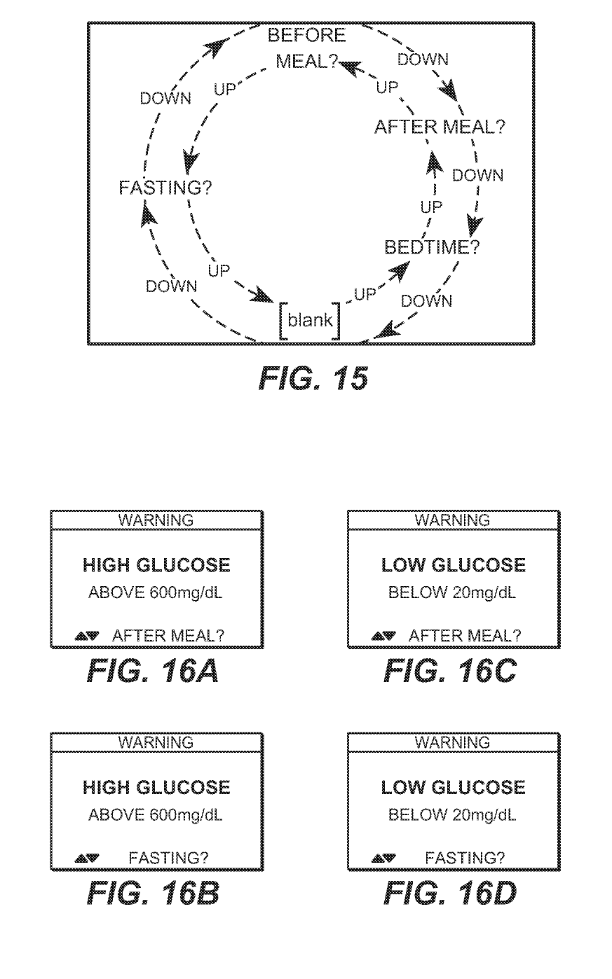

Referring to FIG. 15, the flags can be selected by using the up and down keys of the meter. Alternatively, the various flags can be automatically displayed for selection as a default flag depending on when a blood glucose measurement is taken during various time periods in a day. For example, in one embodiment, a “fasting” flag can be set as a default flag automatically whenever a measurement is taken in the early morning period as determined by the internal clock of the meter 100. A “before meal” flag can be the default flag displayed upon the measurement around certain time periods near meal times. Likewise, an “after meal” flag can be set to be displayed as a default flag for selection by the user whenever a measurement is taken at certain times of the day. A “Bedtime” flag can be set as a default flag automatically whenever a measurement is taken in the late evening as determined by the internal clock of the meter 100.

Referring to FIGS. 16A and 16B, where a measurement exceeds a certain range, a warning message can be displayed and a flag can be associated with such warning message. For example, in FIG. 16A, where the measurement of the analyte exceeds a certain preset value, a warning message of “High Glucose” is displayed. An appropriate flag can be automatically displayed or selected manually by the user as described above. In the example of FIG. 16A, an “After Meal” flag is displayed and a query in the form of a question mark is presented to the user. In FIG. 16B, a “fasting” flag can be displayed with a query for the selection of the flag to be associated with the measurement. FIGS. 16C and 16D illustrate a warning message with examples of the flags that can be associated with a low glucose value. As noted earlier, the time at which such measurement was taken along with the flag selected can be stored in memory for later retrieval by the user or a health care provider for later analysis.

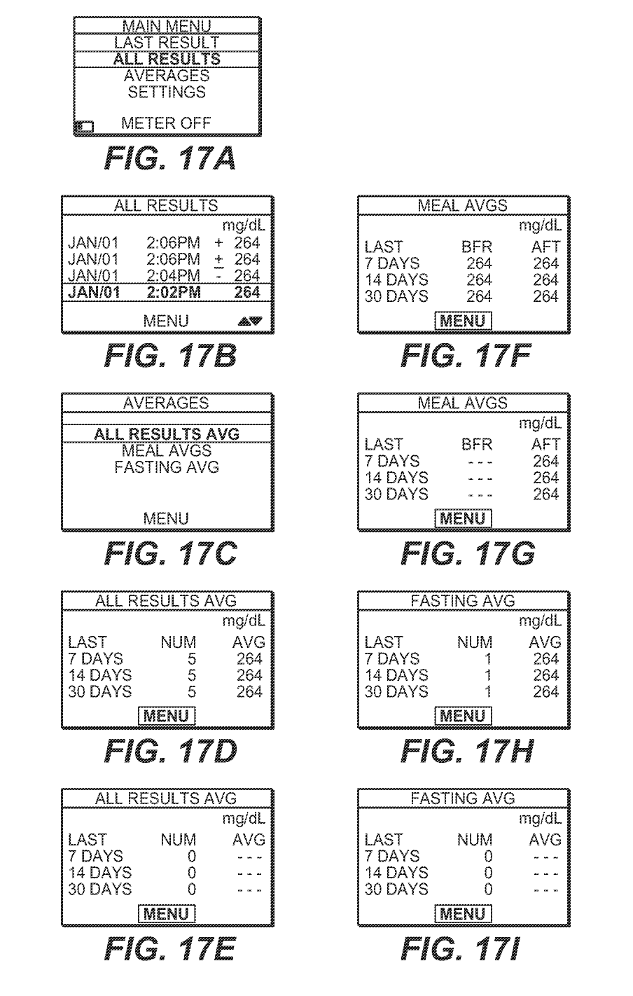

Referring to FIGS. 17A-17I, various screens can be accessed by the users or health care provider to provide statistical data utilized in the treatment of diabetes. As shown in FIG. 17A, a main menu screen allows a user to access various statistical data regarding the blood glucose measurement stored on the meter 100 along with various flags associated therewith, the time, date, year, and any other data useful in the treatment of diabetes.

For example, the meter can be configured to display the following screens in the main menu: “Last Result”; “All Results”; “Averages”; and “Settings.” Where the “Last Result” screen is selected, the meter allows for accessing of the latest result stored in the meter; a selection of “All Results” screen allow for all glucose measurement results stored on the meter to be provided for a complete record to the user, shown here in FIG. 17B where display screen size permitting, four or more results can be displayed at one time; the average of blood glucose data associated with a specific flag can also be obtained with selection of the “Averages” screen.

Referring to FIG. 17C, an “All Results Average” menu can be selected to provide, for example, an average of all blood glucose results stored in the meter. Alternatively, the screen can be configured to provide for a median value (not shown) of the blood glucose value from all of the results stored in the meter instead of an average of all the results. Where this screen is highlighted and selected in FIG. 17C, a screen, shown in FIG. 17D is displayed showing various averages by different categories such as, for example, within the last 3, 7, 14, 21, 30, any desired number of days and the average (or median) of the blood glucose value within each time period (e.g., date time year) and whether such value was before (“BFR”) or after (“AFT”) a meal. Where there are not enough data to display the average in the various time periods, the display will shown, as in FIG. 17E, dashed lines indicating insufficient data.

Referring to FIG. 17C where the “Meal Averages” screen is selected, the display is configured to display, as shown here in FIG. 17F of the meal averages (or median) of the measured glucose value by different time periods and whether the average was before or after a meal. Again, where there is insufficient data, the screen will display dashed lines indicating the same in FIG. 17G.

The fasting average of blood glucose measured can also be obtained by selecting the “Fasting Average” screen in FIG. 17C by the user, which would then be shown in FIG. 17H in various time periods. As before, the meter can display the median instead of average glucose value. Where there is insufficient data, the display will indicate the same by a series of dashed lines as shown in FIG. 17I.

Now that several methods have been described for performing a glucose test, the following will describe methods of detecting a pattern for fasting glucose measurements. Fasting glucose measurements can be important for determining a user's diabetes disease state. Fasting glucose concentrations or trends can be used for determining an insulin dosage amount, an acceptable level of exercise activity, or an amount of food to eat.

FIG. 18 illustrates an exemplary flow chart of a method 1600 for detecting a significant change in fasting glucose concentrations for two time periods. A microprocessor can be programmed to generally carry out the steps of method 1600. The microprocessor can be part of a particular device, such as, for example, a glucose meter, an insulin pen, an insulin pump, a server, a mobile phone, personal computer, or mobile hand held device. A number of glucose measurements can be performed during a first time period via a glucose meter, as shown in a step 1602. Note that each glucose measurement can be associated with a date and time of when the test occurred, and also with a fasting flag when the user had not recently eaten. In an embodiment, fasting may be defined as a glucose measurement performed more than about 8 hours to about 10 hours after eating a meal. The glucose meter can transfer (i.e., upload) data acquired during the first time period to a DMU such as, for example, a mobile computing device (e.g., mobile phone or smart phone) or computer 26, as shown in a step 1604. Next, a number of glucose measurements can be performed during a second time period via the glucose meter, as shown in a step 1606. The glucose meter can then transfer data acquired during the second time period to a DMU, as shown in a step 1608 for subsequent analysis and display on the DMU, as further described herein. Alternatively, the glucose meter itself can perform such data analysis and provide the results to the user via the display of the glucose meter.

Note that steps 1604 and 1608 can be optional where the method is performed without a DMU. In such an embodiment, all of the glucose data would be on the glucose meter, but would be parsed into two time periods, which can be defined by the user or be a default setting.

A check can be performed to determine whether a mixed date condition exists, as shown in a step 1610. Normally, a series of successively saved glucose readings should have time stamps (i.e., date and time) in chronological order. A mixed date condition refers to a situation where one of the successively saved measurements has a time stamp that does not follow a chronological order. In such a scenario, the most recently tested glucose measurement can have a time stamp that is earlier than the time stamp of the immediately previous measurement. The mixed date condition can cause glucose measurements to have the appearance of being back-dated. A mixed date condition may arise when a user does not properly set the clock after a condition such as replacing a battery. If a mixed date condition is detected, method 1800 can be initiated without providing a message that the fasting glucose concentrations has significantly increased or decreased for the first and second time period. Alternatively, both methods 1600 and 1800 can be stopped when a mixed date condition is identified. An embodiment of a method for identifying a mixed date condition can be found in U.S. Pre-Grant Publication No. 2008/0194934, which is hereby fully incorporated by reference herein with a copy attached hereto this application.

Once the mixed date condition test is performed, the number of fasting flags that occurred during the first and second time periods (N₁ and N₂) can be calculated and compared to a threshold, as shown in a step 1612. Method 1600 can be allowed continue where the number of the fasting flags during the first time period N₁ and the second time period N₂ are each greater than 10. Otherwise, method 1800 can be initiated without providing a message that the fasting glucose concentrations has significantly increased or decreased for the first and second time period.

A chi-squared table can be generated, as shown in a step 1616, where both N₁ and N₂ are greater than 10. In the chi-squared table, a row can be represented by a Condition i and a column can be represented by an Outcome 1 or 2. For method 1600, Condition 1 represents the glucose measurements during the first time period, Condition 2 represents the glucose measurements during the second time period, Outcome 1 represents the number of fasting glucose concentrations above the overall median, and Outcome 2 represents the number of fasting glucose concentrations below or equal to the overall median. Note that fasting glucose concentrations can be defined as glucose measurements having an associated fasting flag.

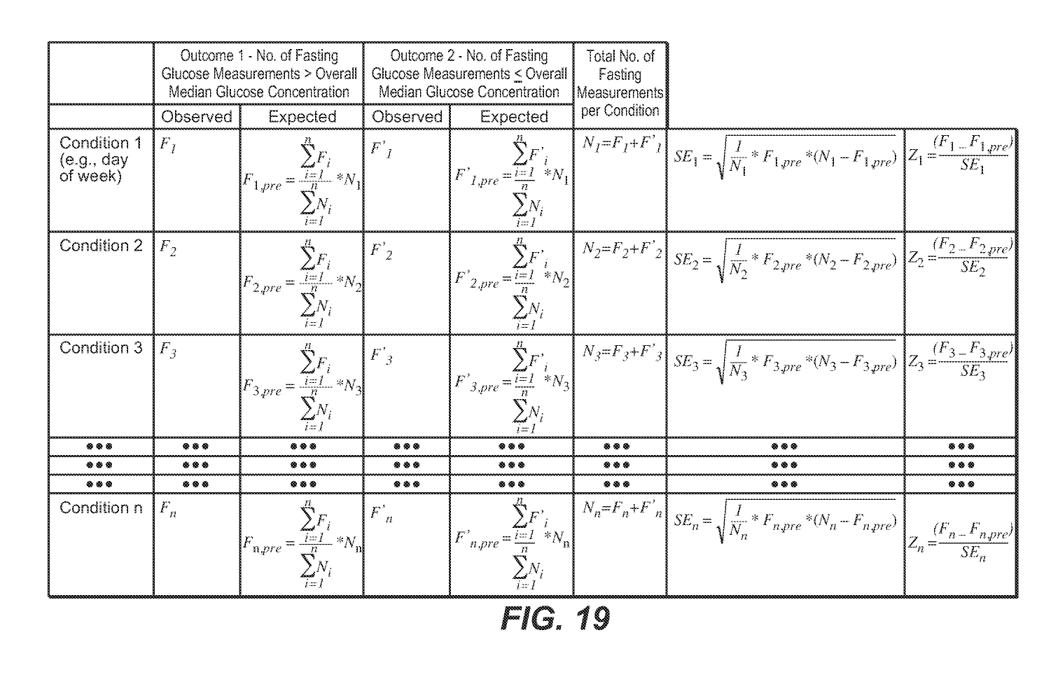

The following will describe in more details the “observed” terms in the table of FIG. 19. F₁ represents the observed number of fasting glucose concentrations during the first time period above the overall median. The overall median is the median value of all glucose concentrations from the first and second time periods. F′₁ represents the observed number of fasting glucose concentrations during the first time period below or equal to the overall median. F₂ represents the observed number of fasting glucose concentrations during the second time period above the overall median. F′₂ represents the observed number of fasting glucose concentrations during the second time period below or equal to the overall median.

The following will describe in more details the “expected” terms in the table of FIG. 19. F_{1,pre} represents the expected number of fasting glucose concentrations during the first time period above the overall median. The overall median is the median value of all glucose concentrations from the first and second time periods. F′_{1,pre} represents the expected number of fasting glucose concentrations during the first time period below or equal to the overall median. F_{2,pre} represents the expected number of fasting glucose concentrations during the second time period above the overall median. F′_{2,pre} represents the expected number of fasting glucose concentrations during the second time period below or equal to the overall median.

Referring back to FIG. 19, the term F_{1,pre} can be calculated using Equation 1 where i=1. Note that the term F_{2,pre} can be calculated using Equation 1 where i=2.

$${F}_{i , pre} = \frac{{\sum }_{i = 1}^{n} {F}_{i}}{{\sum }_{i = 1}^{n} {N}_{i}} * {N}_{i} \qquad Eq . 1$$

The numerator term

$${\sum }_{i = 1}^{n} {F}_{i}$$

can represent the total number of observed flagged glucose measurements greater than the overall median for the first and second time period time period where n=2. The denominator term

$${\sum }_{i = 1}^{n} {N}_{i}$$

can represent the total number of flagged glucose measurements for the first and second time period time period where n=2. As mentioned earlier, the term N₁ represents the total number of flagged glucose measurements during the first time period. N₁ can also be represented as F₁+F′₁.

Referring back again to FIG. 19, the term F′_{1,pre} can be calculated using Equation 2 where i=1. Note that the term F′_{2,pre} can be calculated using Equation 2 where i=2.

$${F}_{i , pre}^{′} = \frac{{\sum }_{i = 1}^{n} {F}_{i}^{′}}{{\sum }_{i = 1}^{n} {N}_{i}} ⋆ {N}_{i} \qquad Eq . 2$$

The numerator term

$${\sum }_{i = 1}^{n} {F}_{i}^{′}$$

can represent the total number of observed flagged glucose measurements less than or equal to the overall median for the first and second time period time period where n=2.

Once the chi-squared table is generated, a step 1618 can be performed to determine whether each of the terms F_{i,pre} and F′_{i,pre} are not less than five and not equal to zero (for i=1 to 2). Note that the terms SE and Z-Test columns of the table in FIG. 19 will be described below for use in method 1800. If one of the terms F_{i,pre} or F′_{i,pre} is equal to zero, this indicates that the particular time period has flagged glucose concentrations that either are all greater than the overall median, or alternatively, not greater than the overall median. In such a case, there is no need to perform a statistical test to determine a significant increase or decrease in fasting glucose concentration. If the F_{i,pre} and F′_{i,pre} are not less than five and not equal to zero, then the method can move to a step 1620. Otherwise, method 1600 can move to method 1800.

In step 1620, a chi-squared value can be calculated using a degree-of-freedom=1. The chi-squared test can be used to determine whether the first and second time periods are statistically different from each other. The chi-squared test may use a confidence level ranging from about 95% to about 99%. Equation 3 shows an example of how to calculate chi-squared X².

$${χ}^{2} = {\sum }_{i = 1}^{n} \frac{{( {F}_{i} - {F}_{i , pre} )}^{2}}{{F}_{i . pre}} + {\sum }_{i = 1}^{n} \frac{{( {F}_{i}^{′} - {F}_{i , pre}^{′} )}^{2}}{{F}_{i . pre}^{′}} \qquad Eq . 3$$

Note that the terms in Equation 3 have been previously described in the table of FIG. 19. After determining X² using Equation 3, the calculated X² value is compared to a X² value in a statistical reference table (degree-of-freedom=1). If the calculated X² value is greater than the X² value on the table, then the two time periods are statistically different and the method can move to a step 1624. If the calculated X² is not greater than the X² value on the table, then the method can move to method 1800. In an embodiment, a significant difference can be a statistical difference.

After determining that there is a significant (or alternatively, a statistical) difference, a calculation can be performed to determine whether a second median M₂ of the flagged glucose concentrations during the second time period is greater than a first median M₁ of the flagged glucose concentrations during the first time period, as shown in step 1624. If M₂ is greater than M₁, then a warning can be outputted via the DMU or on the glucose meter that the fasting glucose concentration has significantly increased for the second or most recent time period, as shown in a step 1626. If M₂ is not greater than M₁, then a warning can be outputted via a display of the DMU or the glucose meter that the fasting glucose concentration has significantly decreased for the second or most recent time period, as shown in a step 1628. Method 1800 can then be initiated after either of steps 1626 or 1628.

FIG. 20 illustrates an exemplary flow chart of method 1800 for detecting a significant change in fasting glucose concentrations for a day of the week. A microprocessor can be programmed to generally carry out the steps of method 1800. The microprocessor can be part of a particular device, such as, for example, a glucose meter, an insulin pen, an insulin pump, a server, a mobile phone, personal computer, or mobile hand held device. A number of glucose measurements can be performed over a plurality of weeks, as shown in a step 1802. A glucose meter can transfer data acquired over the plurality of weeks to a DMU such as computer 26, as shown in a step 1804.

A check can be performed to determine whether a mixed date condition exists, as shown in a step 1810. Method 1800 can be aborted if a mixed date condition is detected. Once the mixed date condition test is performed, the number of fasting flags that occurred during plurality of weeks can be determined and compared to a threshold, as shown in a step 1812. The method 1800 can be allowed continue where the number of the fasting flags during the plurality of weeks N_{W} is greater than 47. Otherwise, method 1800 can be aborted without providing a message comparing the fasting glucose concentration by the days of the week, as shown in a step 1814.

A chi-squared table can be generated, as shown in a step 1816, where N_{W} is greater than 47. Referring back to the chi-squared table of FIG. 19 and applying it to method 1800, Conditions 1 to 7 can represent the glucose measurements performed on a particular day of the week (e.g., 1=Monday to 7=Sunday). Outcome 1 can represent the number of fasting glucose concentrations above the overall median, and Outcome 2 can represent the number of fasting glucose concentrations below or equal to the overall median.

The following will describe in more details the “observed” terms for method 1800 using the table of FIG. 19. F; can represent the observed number of fasting glucose concentrations performed on a particular day of the week (e.g., i=1 to 7) that were above the overall median. Here, the overall median is the median value of all N_{W}, glucose concentrations. F′ᵢ can represent the observed number of fasting glucose concentrations performed on a particular day of the week (e.g., i=1 to 7) that were below or equal to the overall median.

The following will describe in more details the “expected” terms for method 1800 using the table of FIG. 19. F_{i,pre} can represent the expected number of fasting glucose concentrations performed on a particular day of the week (e.g., i=1 to 7) that were above the overall median. F′_{i,pre} can represent the expected number of fasting glucose concentrations performed on a particular day of the week (e.g., i=1 to 7) that were below or equal to the overall median.

Once the chi-squared table is generated, a step 1818 can be performed to determine whether each of the terms F_{i,pre} and F′_{i,pre} are not less than five and not equal to zero (for i=1 to 7). If the F_{i,pre} and F′_{i,pre} are not less than five and not equal to zero, then the method can move to a step 1820. Otherwise, method 1800 can be stopped without generating a message, as shown in step 1814.

In step 1820, a chi-squared value can be calculated using Equation 3 and a degree-of-freedom value=n−C−1. Note that n can be 7 to represent the days of the week. C can represent the number of days of the week in which no glucose readings were performed. Method 1800 can still be performed if there is a particular day or days of the week that do not have any fasting glucose readings. However, if a day of the week is omitted from the analysis of method 1800, a qualifying message will be provided to the user that certain day(s) are missing.

After determining X², the calculated X² value is compared to a X² value in a statistical reference table based on the number of degrees of freedom, as shown in a step 1822. If the calculated X² value is greater than the X² value on the table, then at least one of the days of the week is statistically different and the method can move to a step 1823. If the calculated X² is not greater than the X² value on the table, then the method can be stopped without generating a message, as shown in step 1814.

A standard error SE and a Z test can be calculated for each day of the week, as shown in a step 1823 (see FIG. 19). The Z test can be performed for each day of the week to determine whether a particular day has a statistical difference from the other days of the week. The standard error SE is needed as an intermediate term for performing a Z test. The standard error SE can be calculated for each day i using Equation 4.

$${SE}_{i} = \sqrt{\frac{1}{{N}_{i}} ⋆ {F}_{i , pre} ⋆ ( {N}_{i} - {F}_{i , pre} )} \qquad Eq . 4$$

A Zᵢ value may be calculated for each day i using Eq. 5.

$${Z}_{i} = \frac{( {F}_{i} - {F}_{i , pre} )}{{SE}_{i}} \qquad Eq . 5$$

The calculated Zᵢ value can be compared to a Z value in a statistical reference table, as shown in steps 1824 and 1825. If the Zᵢ value for one of the days is greater than 2, as shown in step 1824, then output a message that the fasting glucose concentration is statistically higher for that particular day, as shown in a step 1826. If the Zᵢ value for one of the days is less than −2, as shown in step 1825, then output a message that the fasting glucose concentration is statistically lower for that particular day, as shown in a step 1828. If the Zᵢ value for all of the days is not greater than 2 and not less than −2, then the method can be stopped without generating a message, as shown in step 1814. Note the message in either step 1826 or 1828 can be qualified to indicate that there was no data for a certain day or days of the week.

Now that methods of detecting a pattern for fasting glucose measurements have been described, the following will describe methods of detecting a pattern for bedtime glucose measurements. Bedtime glucose measurements can be important for determining the appropriate medication or food intake before going to bed. Since the user will not be conscious for several hours while sleeping, it is important that a user have a sufficiently high glucose concentration. Death can easily occur if a user becomes hypoglycemic while sleeping.

FIG. 21 illustrates an exemplary flow chart of a method 2100 for detecting a significant change in bedtime glucose concentrations for two time periods. Method 2100 can be performed after method 1800 is performed. A number of glucose measurements can be performed during a first time period via a glucose meter, as shown in a step 2102. Note that each glucose measurement can be associated with a date and time of when the test occurred, and also with a bedtime flag when the user goes to bed soon after the test. In an embodiment, bedtime may be defined as a glucose measurement performed just before the user goes to sleep for the evening such as, for example, less than about 1 hour before going to bed. In an alternative embodiment, a bedtime flag can be suggested for glucose measurements performed during a predetermined time period programmed into the meter by either a user or a meter manufacturer. A glucose meter can transfer (i.e., upload) data acquired during the first time period to a DMU such as computer 26, as shown in a step 2104. Next, a number of glucose measurements can be performed during a second time period via the glucose meter, as shown in a step 2106. The glucose meter can then transfer data acquired during the second time period to a DMU, as shown in a step 2108 for subsequent analysis and display on the DMU, as further described herein. Alternatively, the glucose meter itself can perform such data analysis and provide the results to the user via the display of the glucose meter.

Note that steps 2104 and 2108 can be optional where the method is performed without a DMU. In such an embodiment, all of the glucose data would be on the glucose meter, but would be parsed into two time periods, which can be defined by the user or be a default setting.

A check can be performed to determine whether a mixed date condition exists, as shown in a step 2110. If a mixed date condition is detected, method 2300 can be initiated without providing a message that the bedtime glucose concentrations has significantly increased or decreased for the first and second time period. Alternatively, both methods 2100 and 2300 can be stopped when a mixed date condition is identified.

Once the mixed date condition test is performed, the number of bedtime flags that occurred during the first and second time periods (N₁ and N₂) can be calculated and compared to a threshold, as shown in a step 2112. Method 2100 can be allowed continue where the number of the bedtime flags during the first time period N₁ and the second time period N₂ are each greater than 10. Otherwise, method 2300 can be initiated without providing a message that the bedtime glucose concentrations has significantly increased or decreased for the first and second time period.

A chi-squared table can be generated, as shown in a step 2116, where both N₁ and N₂ are greater than 10. In the chi-squared table, a row can be represented by a Condition i and a column can be represented by an Outcome 1 or 2. For method 2100, Condition 1 represents the glucose measurements during the first time period, Condition 2 represents the glucose measurements during the second time period, Outcome 1 represents the number of bedtime glucose concentrations above the overall median, and Outcome 2 represents the number of bedtime glucose concentrations below or equal to the overall median. Note that bedtime glucose concentrations can be defined as glucose measurements having an associated bedtime flag.

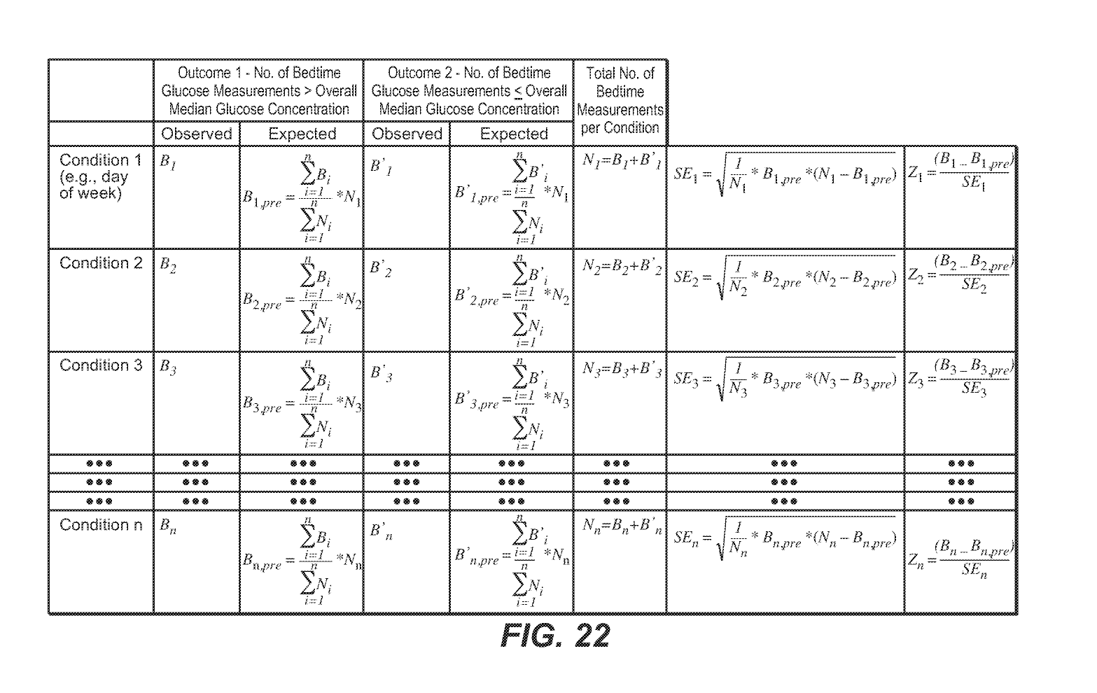

The following will describe in more details the “observed” terms in the table of FIG. 22. B₁ represents the observed number of bedtime glucose concentrations during the first time period above the overall median. The overall median is the median value of all glucose concentrations from the first and second time periods. B′₁ represents the observed number of bedtime glucose concentrations during the first time period below or equal to the overall median. B₂ represents the observed number of bedtime glucose concentrations during the second time period above the overall median. B′₂ represents the observed number of bedtime glucose concentrations during the second time period below or equal to the overall median.

The following will describe in more details the “expected” terms in the table of FIG. 22. B_{1,pre} represents the expected number of bedtime glucose concentrations during the first time period above the overall median. The overall median is the median value of all glucose concentrations from the first and second time periods. B′_{1,pre} represents the expected number of bedtime glucose concentrations during the first time period below or equal to the overall median. B_{2,pre} represents the expected number of bedtime glucose concentrations during the second time period above the overall median. B′_{2,pre} represents the expected number of bedtime glucose concentrations during the second time period below or equal to the overall median.

Referring back to FIG. 22, the term B_{1,pre} can be calculated using Equation 6 where i=1. Note that the term B_{2,pre} can be calculated using Equation 6 where i=2.

$${B}_{i , pre} = \frac{{\sum }_{i = 1}^{n} {B}_{i}}{{\sum }_{i = 1}^{n} {N}_{i}} ⋆ {N}_{i} \qquad Eq . 6$$

The numerator term

$${\sum }_{i = 1}^{n} {B}_{i}$$

can represent the total number of observed flagged glucose measurements greater than the overall median for the first and second time period time period where n=2. The denominator term

$${\sum }_{i = 1}^{n} {N}_{i}$$

can represent the total number of flagged glucose measurements for the first and second time period time period where n=2. As mentioned earlier, the term N₁ represents the total number of flagged glucose measurements during the first time period. N₁ can also be represented as B₁+B′₁.

Referring back again to FIG. 22, the term B′_{1,pre} can be calculated using Equation 7 where i=1. Note that the term B′_{2,pre} can be calculated using Equation 7 where i=2.

$${B}_{i , pre}^{′} = \frac{{\sum }_{i = 1}^{n} {B}_{i}^{′}}{{\sum }_{i = 1}^{n} {N}_{i}} ⋆ {N}_{i} \qquad Eq . 7$$

The numerator term

$${\sum }_{i = 1}^{n} {B}_{i}^{′}$$

can represent the total number of observed flagged glucose measurements less than or equal to the overall median for the first and second time period time period where n=2.

Once the chi-squared table is generated, a step 2118 can be performed to determine whether each of the terms B_{i,pre} and B′_{i,pre} are not less than five and not equal to zero (for i=1 to 2). Note that the terms SE and Z-Test columns of the table in FIG. 22 will be described below for use in method 2300. If one of the terms B_{i,pre} or B′_{i,pre} is equal to zero, this indicates that the particular time period has flagged glucose concentrations that either are all greater than the overall median, or alternatively, not greater than the overall median. In such a case, there is no need to perform a statistical test to determine a significant increase or decrease in bedtime glucose concentration. If the B_{i,pre} and B_{i,pre} are not less than five and not equal to zero, then the method can move to a step 2120. Otherwise, method 2100 can move to method 2300.

In step 2120, a chi-squared value can be calculated using a degree-of-freedom=1. The chi-squared test can be used to determine whether the first and second time periods are statistically different from each other. The chi-squared test may use a confidence level ranging from about 95% to about 99%. Equation 8 shows an example of how to calculate chi-squared X².

$${χ}^{2} = {\sum }_{i = 1}^{n} \frac{{( {B}_{i} - {B}_{i , pre} )}^{2}}{{B}_{i . pre}} + {\sum }_{i = 1}^{n} \frac{{( {B}_{i}^{′} - {B}_{i , pre}^{′} )}^{2}}{{B}_{i . pre}^{′}} \qquad Eq . 8$$

Note that the terms in Equation 8 have been previously described in the table of FIG. 22. After determining X² using Equation 8, the calculated X² value is compared to a X² value in a statistical reference table (degree-of-freedom=1). If the calculated X² value is greater than the X² value on the table, then the two time periods are statistically different and the method can move to a step 2124. If the calculated X² is not greater than the X² value on the table, then the method can move to method 2300. In an embodiment, a significant difference can be a statistical difference.

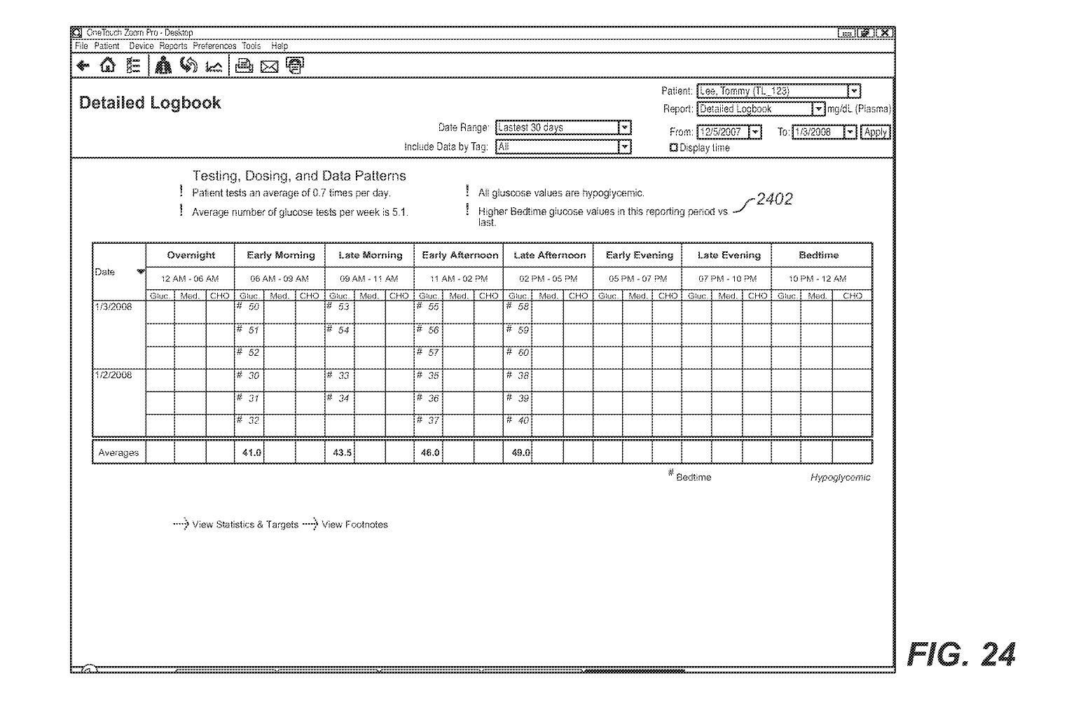

After determining that there is a significant difference (or alternatively, a statistical difference), a calculation can be performed to determine whether a second median M₂ of the flagged glucose concentrations during the second time period is greater than a first median M₁ of the flagged glucose concentrations during the first time period, as shown in step 2124. If M₂ is greater than M₁, then a warning can be outputted via the DMU or on the glucose meter that the bedtime glucose concentration has significantly increased for the second or most recent time period, as shown in a step 2126. An exemplary output on a portion 2402 of a report can illustrate there was a significant increase in bedtime glucose concentrations from the previous periods, as shown in the screen shot of FIG. 24. If M₂ is not greater than M₁, then a warning can be outputted via a display of the DMU or the glucose meter that the bedtime glucose concentration has significantly decreased for the second or most recent time period, as shown in a step 2128. Method 2300 can then be initiated after either of steps 2126 or 2128.

FIG. 23 illustrates an exemplary flow chart of method 2300 for detecting a significant change in bedtime glucose concentrations for a day of the week. A microprocessor can be programmed to generally carry out the steps of method 2300. The microprocessor can be part of a particular device, such as, for example, a glucose meter, an insulin pen, an insulin pump, a server, a mobile phone, personal computer, or mobile hand held device. A number of glucose measurements can be performed over a plurality of weeks, as shown in a step 2302. A glucose meter can transfer data acquired over the plurality of weeks to a DMU such as computer 26, as shown in a step 2304.

A check can be performed to determine whether a mixed date condition exists, as shown in a step 2310. Method 2300 can be aborted if a mixed date condition is detected. Once the mixed date condition test is performed, the number of bedtime flags that occurred during plurality of weeks can be determined and compared to a threshold, as shown in a step 2312. The method 2300 can be allowed continue where the number of the bedtime flags during the plurality of weeks N_{W} is greater than 47. Otherwise, method 2300 can be aborted without providing a message comparing the bedtime glucose concentration by the days of the week, as shown in a step 2314.

A chi-squared table can be generated, as shown in a step 2316, where N_{W} is greater than 47. Referring back to the chi-squared table of FIG. 22 and applying it to method 2300, Conditions 1 to 7 can represent the glucose measurements performed on a particular day of the week (e.g., 1=Monday to 7=Sunday). Outcome 1 can represent the number of bedtime glucose concentrations above the overall median, and Outcome 2 can represent the number of bedtime glucose concentrations below or equal to the overall median.

The following will describe in more details the “observed” terms for method 2300 using the table of FIG. 22. Bᵢ can represent the observed number of bedtime glucose concentrations performed on a particular day of the week (e.g., i=1 to 7) that were above the overall median. Here, the overall median is the median value of all N_{W}, glucose concentrations. B′ᵢ can represent the observed number of bedtime glucose concentrations performed on a particular day of the week (e.g., i=1 to 7) that were below or equal to the overall median.

The following will describe in more details the “expected” terms for method 2300 using the table of FIG. 22. B_{i,pre} can represent the expected number of bedtime glucose concentrations performed on a particular day of the week (e.g., i=1 to 7) that were above the overall median. B′_{i,pre} can represent the expected number of bedtime glucose concentrations performed on a particular day of the week (e.g., i=1 to 7) that were below or equal to the overall median.

Once the chi-squared table is generated, a step 2318 can be performed to determine whether each of the terms B_{i,pre} and B′_{i,pre} are not less than five and not equal to zero (for i=1 to 7). If the B_{i,pre} and B′_{i,pre} are not less than five and not equal to zero, then the method can move to a step 2320. Otherwise, method 2300 can be stopped without generating a message, as shown in step 2314.

In step 2320, a chi-squared value can be calculated using Equation 8 and a degree-of-freedom value=n−C−1. Note that n can be 7 to represent the days of the week. C can represent the number of days of the week in which no glucose readings were performed. Method 2300 can still be performed if there is a particular day or days of the week that do not have any bedtime glucose readings. However, if a day of the week is omitted from the analysis of method 2300, a qualifying message will be provided to the user that certain day(s) are missing.

After determining X², the calculated X² value is compared to a X² value in a statistical reference table based on the number of degrees of freedom, as shown in a step 2322. If the calculated X² value is greater than the X² value on the table, then at least one of the days of the week is statistically different and the method can move to a step 2323. If the calculated X² is not greater than the X² value on the table, then the method can be stopped without generating a message, as shown in step 2314.

A standard error SE and a Z test can be calculated for each day of the week, as shown in a step 2323 (see FIG. 22). The Z test can be performed for each day of the week to determine whether a particular day has a statistical difference from the other days of the week. The standard error SE is needed as an intermediate term for performing a Z test. The standard error SE can be calculated for each day i using Equation 9.

$${SE}_{i} = \sqrt{\frac{1}{{N}_{i}} ⋆ {B}_{i , pre} ⋆ ( {N}_{i} - {B}_{i , pre} )} \qquad Eq . 9$$

A Zᵢ value may be calculated for each day i using Eq. 10.

$${Z}_{i} = \frac{( {B}_{i} - {B}_{i , pre} )}{{SE}_{i}} \qquad Eq . 10$$

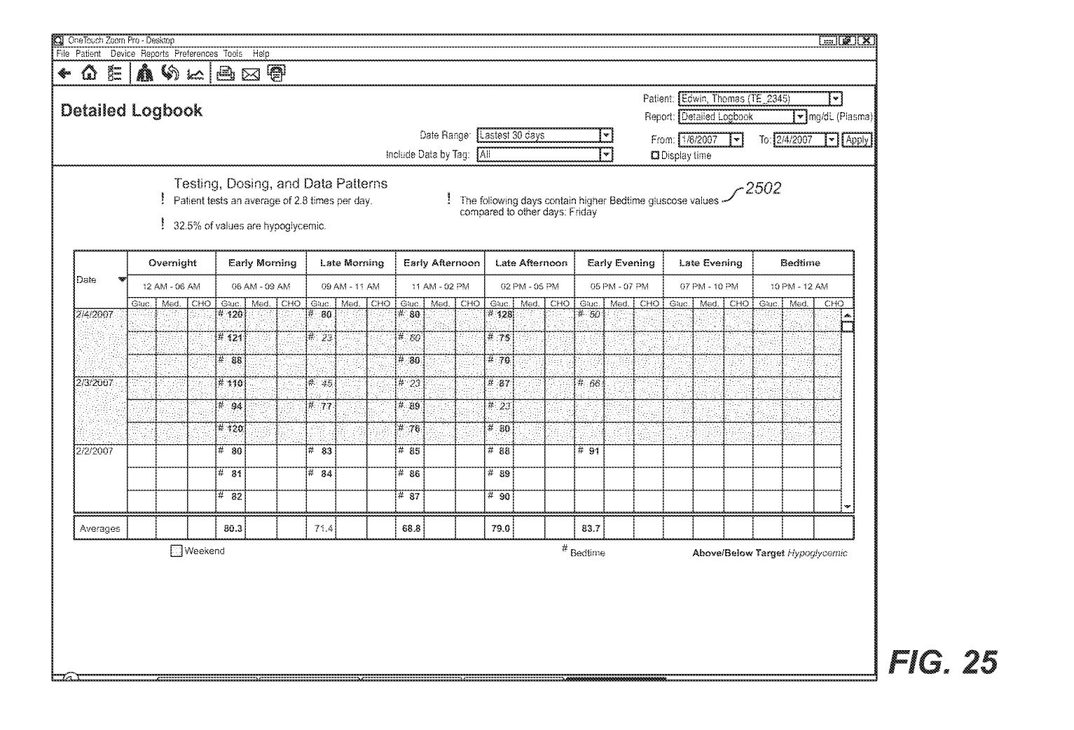

The calculated Zᵢ value can be compared to a Z value in a statistical reference table, as shown in steps 2324 and 2325. If the Zᵢ value for one of the days is greater than 2, as shown in step 2324, then output a message that the bedtime glucose concentration is statistically higher for that particular day, as shown in a step 2326. An exemplary output on a portion 2502 of a report can illustrate there was a significant increase in bedtime glucose concentrations for a particular day of the week such as, for example, Friday, as shown in the screen shot of FIG. 25. If the Zᵢ value for one of the days is less than −2, as shown in step 2325, then output a message that the bedtime glucose concentration is statistically lower for that particular day, as shown in a step 2328. If the Zᵢ value for all of the days is not greater than 2 and not less than −2, then the method can be stopped without generating a message, as shown in step 2314. Note the message in either step 2326 or 2328 can be qualified to indicate that there was no data for a certain day or days of the week.

It is noted that the various methods described herein can be used to generate software codes using off-the-shelf software development tools such as, for example, Visual Studio 6.0, Windows 2000 Server, and SQL Server 2000. The methods, however, may be transformed into other software languages depending on the requirements and the availability of new software languages for coding the methods. Additionally, the various methods described, once transformed into suitable software codes, may be embodied in any computer-readable storage medium that, when executed by a suitable microprocessor or computer, are operable to carry out the steps described in these methods along with any other necessary steps.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

What is claimed is:

1. A diabetes management system comprising:
a plurality of glucose test strips, each test strip configured to receive a physiological sample;
a test strip port connector configured to receive the plurality of test strips; and
a diabetes data management device comprising:
a housing;
a microprocessor coupled to a memory, display, and power supply disposed proximate the housing, the microprocessor coupled to the test strip sensor to provide data representative of a first group and second group of blood glucose values of the user over respective first and second time periods so that respective first and second medians of the first and second group are evaluated by the microprocessor to determine whether one of the first and second medians is significantly different enough to inform the user of the same on the display of the device.

2. The diabetes management system of claim 1, in which the first and second medians are calculated by the microprocessor with glucose values including a common type of flag.

3. The diabetes management system of claim 2, in which the common type of flag comprises at least one of a fasting flag or a bedtime flag.

4. The diabetes management system of claim 1, in which the diabetes data management device comprises a blood glucose meter.

5. The diabetes management system of claim 1, in which the diabetes data management device comprises a combination of a blood glucose meter and mobile phone electronically coupled to each other and in which the blood glucose meter includes the test strip port and a microprocessor to provide blood glucose data to a microprocessor of the mobile phone.

6. A method of detecting a fasting glucose concentration pattern with an analyte testing device having a microprocessor coupled to a memory, the method comprising:
obtaining from the memory of the analyte testing device a first group and second group of glucose measurements over a first time period and a second time period, respectively;
determining whether the fasting glucose concentrations of the first group are significantly different than the fasting glucose concentrations of the second group;
calculating a first median and a second median of the fasting glucose measurements over a first time period and a second time period, respectively;
displaying a message indicating that the second group has a significantly higher fasting glucose concentration than the first group where the second median is greater than the first median, and the first group and second group are significantly different; and
displaying a message indicating that the second group has a significantly lower fasting glucose concentration than the first group where the second median is less than the first median, and the first group and second group are significantly different.

7. A method of detecting a fasting glucose concentration pattern for a day of the week with an analyte testing device having a microprocessor coupled to a memory, the method comprising:
obtaining from the memory a number of glucose measurements over a plurality of weeks, via the analyte testing device;
determining whether the fasting glucose concentrations acquired on at least one day of the week is significantly different than the other days; and
displaying a message indicating that a particular day of the week has a significantly lower or significantly higher fasting glucose concentration than the other days of the week.

8. The method according to claim 7, in which the significant difference includes a statistical difference.

9. The method according to claim 7, in which the determining comprises calculating a chi-squared value using a chi-squared test.

10. The method of claim 9, in which the calculating of the chi-squared value comprises the following equation:
$${χ}^{2} = {\sum }_{i = 1}^{n} \frac{{( {F}_{i} - {F}_{i , pre} )}^{2}}{{F}_{i \cdot pre}} + {\sum }_{i = 1}^{n} \frac{{( {F}_{i}^{′} - {F}_{i , pre}^{′} )}^{2}}{{F}_{i \cdot pre}^{′}} ,$$
where
Fᵢ is an observed number of fasting glucose concentrations above an overall median during a time period i;
F′ᵢ is an observed number of fasting glucose concentrations below or equal to an overall median during the time period i;
F_{i,pre} is an expected number of fasting glucose concentrations above an overall median during the time period i;
F_{i,pre} is an expected number of fasting glucose concentrations below or equal to the overall median during the time period i; and
n is a number of time periods.

11. The method of claim 9, in which the calculating comprises a determination that at least one of the time periods i is statistically different when the calculated chi-squared value is greater than a reference chi-squared value.

12. The method of claim 6, in which the first group and the second group each have greater than ten fasting glucose concentrations.

13. The method of claim 10, in which F_{i,pre} comprises a value based on the following equation,
$${F}_{i , pre} = \frac{{\sum }_{i = 1}^{n} {F}_{i}}{{\sum }_{i = 1}^{n} {N}_{i}} * {N}_{i} ,$$
where Nᵢ represents a total number of flagged glucose measurements during a time period i.

14. The method of claim 10, in which F′_{i,pre} comprises a value based on the following equation,
$${F}_{i , pre}^{′} = \frac{{\sum }_{i = 1}^{n} {F}_{i}^{′}}{{\sum }_{i = 1}^{n} {N}_{i}} * {N}_{i} ,$$
where Nᵢ represents a total number of flagged glucose measurements during a time period i.

15. A method of detecting a bedtime glucose concentration pattern with an analyte testing device having a microprocessor coupled to a memory, the method comprising:
obtaining from the memory of the analyte testing device a first group and second group of glucose measurements over a first time period and a second time period, respectively;
determining whether the bedtime glucose concentrations of the first group are significantly different than the bedtime glucose concentrations of the second group;
calculating a first median and a second median of the bedtime glucose measurements over a first time period and a second time period, respectively;
displaying a message indicating that the second group has a significantly higher bedtime glucose concentration than the first group where the second median is greater than the first median, and the first group and second group are significantly different; and
displaying a message indicating that the second group has a significantly lower bedtime glucose concentration than the first group where the second median is less than the first median, and the first group and second group are significantly different.

16. A method of detecting a bedtime glucose concentration pattern for a day of the week with an analyte testing device having a microprocessor coupled to a memory, the method comprising:
obtaining from the memory a number of glucose measurements over a plurality of weeks, via the analyte testing device;
determining whether the bedtime glucose concentrations acquired on at least one day of the week is significantly different than the other days; and
displaying a message indicating that a particular day of the week has a significantly lower or significantly higher bedtime glucose concentration than the other days of the week.

17. The method according to claim 16, in which the significant difference includes a statistical difference.

18. The method according to claim 16, in which the determining comprises calculating a chi-squared value using a chi-squared test.

19. The method of claim 18, in which the calculating of the chi-squared value comprises the following equation:
$${χ}^{2} = {\sum }_{i = 1}^{n} \frac{{( {B}_{i} - {B}_{i , pre} )}^{2}}{{B}_{i \cdot pre}} + {\sum }_{i = 1}^{n} \frac{{( {B}_{i}^{′} - {B}_{i , pre}^{′} )}^{2}}{{B}_{i \cdot pre}^{′}} ,$$
where
Bᵢ is an observed number of bedtime glucose concentrations above an overall median during a time period i;
B′ᵢ is an observed number of bedtime glucose concentrations below or equal to an overall median during the time period i;
B_{i,pre} is an expected number of bedtime glucose concentrations above an overall median during the time period i;
B_{i,pre} is an expected number of bedtime glucose concentrations below or equal to the overall median during the time period i; and
n is a number of time periods.

20. The method of claim 18, in which the calculating comprises a determination that at least one of the time periods i is statistically different when the calculated chi-squared value is greater than a reference chi-squared value.

21. The method of claim 15, in which the first group and the second group each have greater than ten bedtime glucose concentrations.

22. The method of claim 19, in which B_{i,pre} comprises a value based on the following equation,
$${B}_{i , pre} = \frac{{\sum }_{i = 1}^{n} {B}_{i}}{{\sum }_{i = 1}^{n} {N}_{i}} * {N}_{i} ,$$
where Nᵢ represents a total number of flagged glucose measurements during a time period i.

23. The method of claim 19, in which B′_{i,pre} comprises a value based on the following equation,
$${B}_{i , pre}^{′} = \frac{{\sum }_{i = 1}^{n} {B}_{i}^{′}}{{\sum }_{i = 1}^{n} {N}_{i}} * {N}_{i} ,$$
where Nᵢ represents a total number of flagged glucose measurements during a time period i.
